(19) United States
(12) Patent Application Publication
(10) Pub. No.: US 2014/0228389 A1
(43) Pub. Date: Aug. 14, 2014

(54) DRY POWDER COMPOUND FORMULATIONS AND USES THEREOF

(71) Applicant: Wyeth, LLC, Madison, NJ (US)

(72) Inventors: Syed M. Shah, Delray Beach, FL (US); Christian Ofslager, Newburgh, NY (US)

(73) Assignee: WYETH, LLC, Madison, NJ (US)

(21) Appl. No.: 14/027,736

(22) Filed: Sep. 16, 2013

Related U.S. Application Data
(63) Continuation of application No. 13/230,167, filed on Sep. 12, 2011, now abandoned.
Application No. 13/230,167 is a continuation of application No. 11/899,724, filed on Sep. 7, 2007, now abandoned.
(60) Provisional application No. 60/843,437, filed on Sep. 8, 2006.

(51) Int. Cl. A61K 31/485; A61K 9/19; A61K 47/26

(52) U.S. Cl. CPC A61K 31/485; A61K 47/26; A61K 9/19
USPC 514/282

(57) ABSTRACT

The present invention provides lyophilized formulations comprising methylnaltrexone, and processes for preparation of provided formulations. Additionally provided are compositions and products containing the methylnaltrexone formulation, as well as methods for producing formulations, compositions and products. Provided formulations as well as compositions and products containing methylnaltrexone formulations are useful for preventing, treating delaying, diminishing or reducing the severity and/or incidence of side effects resulting from administration of analgesic opioids.

### CROSS-REFERENCE TO RELATED APPLICATIONS

The present invention claims priority to U.S. provisional patent application Ser. No. 60/843,437, filed Sep. 8, 2006, the entirety of which is hereby incorporated herein by reference.

### BACKGROUND OF THE INVENTION

Opioids are widely used in patients with advanced cancers and other terminal diseases to lessen suffering. Opioids are narcotic medications that activate opioid receptors located in the central nervous system to relieve pain. Opioids, however, also react with receptors outside of the central nervous system, resulting in side effects including constipation, nausea, vomiting, urinary retention and severe itching. Most notable are the effects in the gastrointestinal tract (GI) where opioids inhibit gastric emptying and propulsive motor activity of the intestine, thereby decreasing the rate of intestinal transit which can produce constipation. The effectiveness of opioids for pain is often limited due to resultant side effects, which can be debilitating and often cause patients to cease administration of opioid analgesics.

In addition to analgesic opioid induced side effects, studies have suggested that endogenous opioid compounds and receptors may also affect activity of the gastrointestinal (GI) tract and may be involved in normal regulation of intestinal motility and mucosal transport of fluids in both animals and man. (Koch, T. R, et al., Digestive Diseases and Sciences 1991, 36, 712-728; Schuller, A. G. P., et al., Society of Neuroscience Abstracts 1998, 24, 524, Reisine, T., and Pasternak, G., Goodman & Gilman's The Pharmacological Basis of Therapeutics Ninth Edition 1996, 521-555 and Bagnol, D., et al., Regul. Pept. 1993, 47, 259-273). Thus, an abnormal physiological level of endogenous compounds and/or receptor activity may lead to bowel dysfunction.

For example, patients who have undergone surgical procedures, especially surgery of the abdomen, often suffer from bowel dysfunction, such as post-operative (or post-surgical) ileus, that may be caused by fluctuations in natural opioid levels. Similarly, women who have recently given birth commonly suffer from post-partum ileus, which is thought to be caused by similar natural opioid fluctuations as a result of birthing stress. Bowel dysfunction associated with post-operative or post partum ileus can typically last for 3 to 5 days, with some severe cases lasting more than a week. Administration of opioid analgesics to a patient after surgery, which is now an almost universal practice, may exacerbate bowel dysfunction, thereby delaying recovery of normal bowel function, prolonging hospital stays, and increasing medical care costs.

Opioid antagonists such as naloxone, naltrexone, and nalmefene, have been studied as a means of antagonizing undesirable peripheral effects of opioids. However, these agents act not only on peripheral opioid receptors, but also on central nervous system sites, so that they sometimes reverse the beneficial analgesic effects of opioids, or cause symptoms of opioid withdrawal. Preferable approaches for use in controlling opioid-induced side effects include administration of peripheral opioid antagonist compounds that do not readily cross the blood-brain barrier. For example, the peripheral g opioid antagonist compound methylnaltrexone and related compounds have been disclosed for use in curbing opioid-induced side effects in patients (e.g., constipation, pruritus, nausea, and/or vomiting). See, e.g., U.S. Pat. Nos. 5,972,954, 5,102,887, 4,861,781, and 4,719,215; and Yuan, C.-S. et al. Drug and Alcohol Dependence 1998, 52, 161.

Formulations of peripheral μ opioid receptor antagonist methylnaltrexone have been described (e.g., see, for example, U.S. Pat. Nos. 6,608,075, 6,274,591, and 6,559,158). However, methylnaltrexone in certain mediums and under certain conditions has been found to form degradation products. For example, see US 2004266806A1. It is desirable to provide dosage forms that are capable of effective delivery of peripheral methylnaltrexone without extensive degradation of the methylnaltrexone under refrigeration and/or room temperature conditions. It is desirable to provide a process for production of a stabilized methylnaltrexone formulation suitable for intravenous administration to a subject in need thereof. It is also desirable to provide a product with solid state stability at room temperature and reconstitution stability for dosing to a subject.

### SUMMARY OF THE INVENTION

The present invention provides dry powder formulations of methylnaltrexone. In some embodiments, provided formulations are a dry powder containing methylnaltrexone and a filler or a cryoprotectant, but lacking other agents typically found in dry powder (e.g., lyophilized) preparations. In some embodiments, provided formulations consist essentially of methylnaltrexone and a single filler or single cryoprotectant. In some embodiments, provided formulations are a dry, amorphous cake. In some embodiments, provided formulations are storage-stabile. In some embodiments, provided formulations are stable to extended storage at room temperature. For example, provided formulations may be storage stable for a period of at least about one month, two months, three months, four months, five months, six months, or more. In some embodiments, provided formulations are storage stable for 12 months or for more than 24 months.

Provided formulations are useful for administration to subjects. For example, in some embodiments, provided formulations are suitable for parenteral administration of methylnaltrexone. In some embodiments, provided formulations contain an amount of methylnaltrexone suitable for single dose administration. In other embodiments, provided formulations contain an amount of methylnaltrexone suitable for multiple dose administration.

The present invention also provides methods for preparing dry powder formulations, as well as liquid formulations reconstituted from or prepared into such dry powder formulations. In some embodiments, dry powder formulations are prepared by lyophilization; in some embodiments dry powder formulations are prepared by spray drying of a super critical solution. In some embodiments, reconstituted formulations may contain an amount of methylnaltrexone appropriate for direct dosing, or may contain an amount of methylnaltrexone appropriate for further dilution (e.g., for intravenous administration). Additionally provided are methods for production and use of formulations, as well as products and kits containing the provided formulations.

In general, provided formulations are useful for preventing, treating, delaying onset of or reducing severity and/or incidence of side effects resulting from use of opioids, including gastrointestinal dysfunction (e.g., constipation, bowel hypomotility, impaction, gastric hypomotility, GI sphincter constriction, increased sphincter tone, inhibition of gastrointestinal motility, inhibition of intestinal motility, inhibition of gastric emptying, delayed gastric emptying, incomplete evacuation, nausea, emesis (vomiting), bloating, abdominal distension), dysphoria, pruritis, urinary retention, depression of respiration, papillary constriction, cardiovascular effects, chest wall rigidity and cough suppression, depression of stress response, and immune suppression associated with use of narcotic analgesia, etc. Additional effects of opioid administration can include, e.g., aberrant migration or proliferation of endothelial cells (e.g., vascular endothelial cells), increased angiogenesis, and increase in lethal factor production from opportunistic infectious agents (e.g., Pseudomonas aeruginosa).

In some embodiments, provided formulations are useful for administration to patients receiving short term opioid treatment (e.g., patients recovering from surgery (abdominal, orthopedic, surgery from trauma injuries etc.), patients recovering from trauma injuries, and/or patients recovering from child birth). In some embodiments, provided formulations are useful for administration to subjects receiving chronic opioid administration (e.g., terminally ill patients receiving opioid therapy (e.g., an AIDS patient, a cancer patient, a cardiovascular patient); subjects receiving chronic opioid therapy for pain management (e.g., back pain); subjects receiving opioid therapy for maintenance of opioid withdrawal). In some embodiments of the invention, provided formulations are useful for administration to patients suffering from paralytic ileus, whether resulting from administration of opioids (typically prolonged or excessive use of opioids), from normal or aberrant activity of endogenous opioids, or from other causes. In some embodiments, paralytic ileus results from peritonitis, pneumonia, pancreatitis, nerve trauma or decreased blood supply to the intestinal wall, metabolic disturbances (e.g., affecting potassium levels), spinal injury, etc.

In some embodiments, provided formulations are useful, for example, in prevention, treatment, delay, or reduction of severity and/or incidence of symptoms associated with disorders or conditions resulting from normal or aberrant activity of endogenous opioids. Such disorders or conditions can include, among others, ileus (e.g., post-partum ileus), post-operative gastrointestinal dysfunction following abdominal surgery (e.g., colectomy (e.g., right hemicolectomy, left hemicolectomy, transverse hemicolectomy, colectomy takedown, low anterior resection) or hernia repair), such as post operative ileus, and idiopathic constipation. In some embodiments, provided formulations are useful in prevention, treatment, delay, or reduction of severity and/or incidence of symptoms associated with conditions including cancers involving angiogenesis, immune suppression, sickle cell anemia, vascular wounds, retinopathy, and treatment of inflammation associated disorders (e.g., irritable bowel syndrome), immune suppression, chronic inflammation.

### DETAILED DESCRIPTION OF CERTAIN EMBODIMENTS OF THE INVENTION

In certain embodiments, the present invention provides pharmaceutical compositions having improved stability characteristics. Opioid antagonist formulations comprising methylnaltrexone are provided which are useful to prevent, treat, delay or reduce the severity and/or incidence of undesirable side effects of opioid administration or activity. In some embodiments, provided compositions, and kits and products including them, allow for extended storage periods and/or for storage under favorable room temperature conditions. Provided compositions, and kits and products containing them, thus allow for improved delivery of therapeutics to subjects benefiting from use of methylnaltrexone.

For example, provided formulations are useful to treat, prevent, delay, or decrease severity and/or incidence of side effects associated with opioid administration, including gastrointestinal dysfunction (e.g., constipation, bowel hypomotility, impaction, gastric hypomotility, GI sphincter constriction, increased sphincter tone, inhibition of gastrointestinal motility, inhibition of intestinal motility, inhibition of gastric emptying, delayed gastric emptying, incomplete evacuation, nausea, emesis (vomiting), bloating, abdominal distension), dysphoria, pruritis, urinary retention, depression of respiration, papillary constriction, cardiovascular effects, chest wall rigidity and cough suppression, depression of stress response, and immune suppression associated with administration of narcotic analgesia, etc. Additional effects of opioid administration can include, e.g., aberrant migration or proliferation of endothelial cells (e.g., vascular endothelial cells), increased angiogenesis, and increase in lethal factor production from opportunistic infectious agents (e.g., Pseudomonas aeruginosa).

In certain embodiments, provided formulations are useful for administration to patients receiving short term treatment with opioids (e.g., patients suffering from post-operative gastrointestinal dysfunction receiving short term opioid administration). In some embodiments, provided formulations are useful for administration to subjects receiving chronic opioid administration (e.g., terminally ill patients receiving opioid therapy such as an AIDS patient, a cancer patient, a cardiovascular patient; subjects receiving chronic opioid therapy for pain management; subjects receiving opioid therapy for maintenance of opioid withdrawal).

Alternatively or additionally, certain provided formulations may be useful, for example, in prevention, treatment, delay, or reduction of severity and/or incidence of symptoms associated with disorders or conditions resulting from normal or aberrant activity of endogenous opioids. Such disorders or condition include, among others, ileus (e.g., post-operative ileus, post-partum ileus, paralytic ileus), post-operative gastrointestinal dysfunction following abdominal surgery (e.g., colectomy (e.g., right hemicolectomy, left hemicolectomy, transverse hemicolectomy, colectomy takedown, low anterior resection) or hernia repair), and idiopathic constipation. In some embodiments of the invention, provided formulations are useful in treatment, prevention, delay, or reduction of severity and/or incidence of side effects in conditions including cancers involving angiogenesis, immune suppression, sickle cell anemia, vascular wounds, and retinopathy, treatment of inflammation associated disorders (e.g., irritable bowel syndrome), immune suppression, chronic inflammation.

### DEFINITIONS

The expression “dosage preparation” refers to the form or context in which a formulation is stored and/or used prior to or during administration to a subject. For example, a “dosage preparation” containing a formulation may constitute or comprise the formulation in the context of a vial or syringe appropriate for storage and/or administration. A dosage preparation may constitute or comprise a formulation in the context of a container which protects the formulation from light (e.g., UV light). Alternatively, a dosage preparation may constitute or comprise a formulation in the context of a container which does not protect the formulation from exposure to light. In some embodiments, a dosage preparation may contain a single unit dosage of methyl naltrexone. In some embodiments, a dosage preparation may contain more or less than a single unit dosage of methylnaltrexone. In some embodiments, a dosage preparation may contain an amount of methylnaltrexone that is a multiple of a unit dosage.

The term “dose-concentrate,” as used herein, refers to a pharmaceutical composition having a concentration of active agent(s) higher than a typical unit dosage concentration administered directly to a subject. A dose-concentrate may be used as provided for administration to a subject, but is generally further diluted to a typical unit dosage concentration in preparation for administration to a subject. The entire volume of a dose-concentrate, or aliquots thereof, may be used in preparing unit dosage(s) for treatment, for example, by the methods provided herein. In some embodiments, a dose-concentrate is about 2 fold, about 5-fold, about 10-fold, about 25-fold, about 50-fold, about 100-fold, or about 200-fold more concentrated than a unit dosage. In certain embodiments, a dose concentrate is about 50-fold, about 100-fold, or about 200-fold more concentrated than a unit dosage. A dose-concentrate may be formed by reconstitution of a dry powder formulation by addition of aqueous solvent to a provided formulation.

The term, “dry powder formulation” or “dry powder composition” refers to a dry, solid composition, and encompasses dried compositions prepared by freeze-drying (e.g., lyophilization) or other appropriate methods (e.g., spray drying, super critical fluid formation, etc.) to achieve production of a dried amorphous cake form. Lyophilization is a process of freeze-drying in which water is sublimed from the product after it is frozen, optionally by applying a vacuum. Specifics of lyophilizing or freeze-drying are known in the art and described, for example, in Remington's Pharmaceutical Sciences, Chapter 84, page 1565, 18^{th} Edition, A. R. Gennaro, Editor, 1990, Mack Publishing Company. Techniques other than lyophilization which may also be used for preparation of dry powder formulation(s) (e.g., dried samples), and particularly for preparation of amorphous dry powder formulations, are known in the art, include, but are not limited to, sterile powder filling of the components, singly, or as a complete blend, spray drying, tray drying, sizing processes including milling and/or screening and precipitation. In certain embodiments, inventive dry powder formulations are in the form of a cake (e.g., an amorphous cake).

As used herein, an “effective amount” of a compound or pharmaceutically acceptable formulation can achieve a desired therapeutic and/or prophylactic effect. In some embodiments, an “effective amount” is at least a minimal amount of a compound, or formulation containing a compound, which is sufficient for treating one or more symptoms of a disorder or condition associated with modulation of peripheral μ opioid receptors, such as side effects associated with opioid analgesic therapy (e.g., gastrointestinal dysfunction (e.g., dysmotility constipation, etc.), nausea, emesis, (e.g., vomiting), etc.). In certain embodiments, an “effective amount” of a compound, or formulation containing a compound, is sufficient for treating symptoms associated with, a disease associated with aberrant endogenous peripheral opioid or μ opioid receptor activity (e.g., idiopathic constipation, ileus, etc.).

The term “formulation”, in general, refers to a preparation that includes at least one pharmaceutically active compound (e.g., at least methylnaltrexone, in any appropriate form) optionally in combination with one or more excipients or other pharmaceutical additives for administration to a subject. In general, particular excipients and/or other pharmaceutical additives are typically selected with the aim of enabling a desired stability, release, distribution and activity of active compound(s) for applications. According to the present invention, formulations that “consist essentially of” methylnaltrexone and a single filler or single cryoprotectant generally include only methylnaltrexone and the single filler or cryoprotectant, potentially in the presence of low level contaminants (e.g., process contaminants), degradation products (particularly of the methylnaltrexone) and/or buffering agents. It is understood in the art that preparation of materials and/or formulations sometimes involves the introduction of unavoidable contaminants; compositions containing such contaminants at sufficiently low levels that relevant characteristics of the overall formulation are not materially affected can be within the scope of the present invention.

The term “stable”, as used herein, refers to a formulation whose composition does not change materially over a selected period of time and under selected conditions. For example, in general, a stable formulation of containing methylnaltrexone does not accumulate methylnaltrexone degradation products to a level above 2% over a designated period of time. The term “subject”, as used herein, means a mammal to whom a formulation or composition comprising a formulation is administered, and includes human and animal subjects, such as domestic animals (e.g., horses, dogs, cats, cows, etc.). In some embodiments, the subject is a primate, a domestic animal, or a human. In some embodiments, the subject is a human.

“Therapeutically active agent” or “active agent” refers to a substance, including a biologically active substance, that is useful for therapy (e.g., human therapy, veterinary therapy), including prophylactic and/or therapeutic treatment. Therapeutically active agents can be organic molecules that are drug compounds, peptides, proteins, carbohydrates, monosaccharides, oligosaccharides, polysaccharides, nucleoprotein, mucoprotein, lipoprotein, synthetic polypeptide or protein, small molecules linked to a protein, glycoprotein, steroid, nucleic acid, DNA, RNA, nucleotide, nucleoside, oligonucleotides, antisense oligonucleotides, lipid, hormone, and vitamin. Alternatively or additionally, therapeutically active agents can be any substance used as a medicine for treatment, prevention, delay, reduction or amelioration of a disease, condition, or disorder. Among therapeutically active agents useful in the formulations of the present invention are opioid antagonist compounds, opioid analgesic compounds, and the like. Further detailed description of agents useful as therapeutically active agents is provided below. The term “therapeutically active agent” can also refer to a first agent that increases the effect or effectiveness of a second agent, for example, by enhancing potency, increasing availability, and/or or reducing adverse effects of a second agent.

The expression “unit dosage” as used herein refers to a physically discrete unit of formulation appropriate for a subject to be treated. It will be understood, however, that total daily usage of the compositions of the present invention will be decided by the attending physician within the scope of sound medical judgment. A specific effective dose level for any particular subject or organism may depend upon a variety of factors including the disorder being treated and the severity and/or incidence of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

### Methylnaltrexone

The present invention provides formulations and dosage preparations for parenteral administration of methylnaltrexone. When a formulation, dosage preparation or method described herein is said to utilize “methylnaltrexone,” it should be understood that any appropriate form of methylnaltrexone (e.g. N-methylnaltrexone and/or any pharmaceutically acceptable salts thereof) having desired activity may be utilized. Methylnaltrexone is described for example in U.S. Pat. Nos. 4,176,186; 4,719,215; 4,861,781; 5,102,887; 5,972,954; 6,274,591; U.S. patent application Nos. 20020028825 and 20030022909; and PCT publication Nos. WO 99/22737 and WO 98/25613; the contents of each of which are hereby incorporated by reference.

In general, pharmaceutically acceptable salts include, but are not limited to, chloride, bromide, iodide, nitrate, sulfate, bisulfate, phosphate, acid phosphate, isonicotinate, acetate, lactate, salicylate, citrate, tartrate, pantothenate, bitartrate, carbonate, ascorbate, succinate, maleate, gentisinate, fumarate, gluconate, glucoronate, saccharate, formate, carboxylate, benzoate, glutamate, sulfonate, methanesulfonate, ethanesulfonate, benzensulfonate, p-toluenesulfonate, selenate, and pamoate (i.e., 1,1′-methylene-bis-(2-hydroxy-3-naphthoate)) salts of compounds. In some embodiments, salts of use in formulations of the invention are those that have been described for methylnaltrexone, e.g., methylnaltrexone bromide, etc. However, the invention is not limited to these specific salts. Other salts (e.g., chloride, sulfate, bisulfate, tartrate, nitrate, citrate, bitartrate, phosphate, malate, maleate, bromide, iodide, fumarate, sulfonate, carboxylate, or succinate salts, etc.) and/or mixtures thereof can be adapted and used in a dose formulation according to the invention so as to achieve an appropriate compound delivery profile of the invention. Alternatively or additionally, peripheral opioid receptor antagonist (e.g., methylnaltrexone) base, chemical and chiral derivatives thereof and salts can be used, as appropriate.

The bromide salt of methylnaltrexone is also referred to, for example, N-methylnaltrexone bromide, N-methylnaltrexone hydrobromide, methylnaltrexone bromide, methylnaltrexone hydrobromide, naltrexone methobromide, N-methylnaltrexone, MNTX, SC-37359, MRZ-2663-BR, and N-cyclopropylmethylnoroxy-morphine-metho-bromide. Methylnaltrexone is available in a powder form from Mallinckrodt Pharmaceuticals, St. Louis, Mo., provided as a white crystalline powder freely soluble in water. Its melting point is 254-256° C.

Methylnaltrexone has chiral centers and can therefore occur as stereochemical isomers by virtue of the substituent placement on those chiral centers. Such stereochemical isomers are within the scope of the compounds contemplated for use in the present formulations. In the compositions and methods of the present invention, compounds employed may be individual stereoisomers, as well as mixtures of stereoisomers. In certain aspects, methods of the present invention utilize compounds which are substantially pure stereoisomers. All tautomers are also intended to be encompassed within the compositions of the present invention.

The terms “R” and “S” are used herein, as commonly used in organic chemistry nomenclature, to denote specific configuration of a chiral center. The term “R” refers to “right” and is used to designate the configuration of a chiral center with a clockwise relationship of group priorities (highest to second lowest) when viewed along the bond toward the lowest priority group. The term “S” or “left” is used to designate the configuration of a chiral center with a counterclockwise relationship of group priorities (highest to second lowest) when viewed along the bond toward the lowest priority group. The priority of groups is based upon their atomic number (heaviest isotope first). A partial list of priorities and a discussion of stereochemistry is contained in the book: The Vocabulary of Organic Chemistry, Orchin, et al., John Wiley and Sons Inc., page 126 (1980), which is incorporated herein by reference in its entirety.

In some embodiments, isolated R—N isomers of methylnaltrexone may be utilized in formulations and methods. As used herein, the designation of “R—N-isomer” of methylnaltrexone refers to such compounds in the (R) configuration with respect to the nitrogen. Isolated isomer compounds include, but are not limited to, R—N isomer methylnaltrexone compounds described in U.S. patent application Ser. No. 11/441,395 filed May 25, 2006, published WO2006/127899, which is hereby incorporated herein by reference. In some embodiments, the active compound is an R—N isomer methylnaltrexone, or a salt thereof. The R—N isomer of methylnaltrexone has been found in U.S. Ser. No. 11/441,395 to be an opioid antagonist.

In some embodiments, isolated S—N isomers of methylnaltrexone may be utilized in formulations and methods. As used herein, the designation of “S—N-isomer” of methylnaltrexone refers to such compounds in the (S) configuration with respect to the nitrogen. Isolated isomer compounds include, but are not limited to, S—N isomer of methylnaltrexone compounds described in U.S. patent application Ser. No. 11/441,452, filed May 25, 2006, published WO2006/127898, which is hereby incorporated by reference. In some embodiments, the active compound is an S—N isomer methylnaltrexone, or a salt thereof. The S—N isomer of methylnaltrexone has been found in U.S. Ser. No. 11/441,452 to be an opioid agonist.

In certain embodiments, the methylnaltrexone utilized in formulations or dosage preparations described herein is a mixture of stereoisomers characterized in that it has opioid antagonistic effect. For example, the methylnaltrexone may be a mixture of R—N and S—N methylnaltrexone such that a mixture itself acts as an antagonist and would be useful for methods of use described herein for opioid antagonists. In certain embodiments, R—N methylnaltrexone is used which is substantially free of S—N methylnaltrexone.

In certain embodiments of the present invention, at least about 99.6%, 99.7%, 99.8%, 99.85%, 99.9%, or 99.95% of methylnaltrexone is in the (R) configuration with respect to nitrogen. Methods for determining the amount of (R)—N-isomer, present in a sample as compared to the amount of (S)—N-isomer present in that same sample, are described in detail in WO2006/127899, the entirety of which is hereby incorporated herein by reference. In other embodiments, methylnaltrexone contains 0.15%, 0.10%, or less (S)—N-isomer.

It will be understood by those skilled in the art that, where reference is made herein to amounts of methylnaltrexone utilized in formulations, dosage preparations, or methods, those amounts may refer to total amount of methylnaltrexone (or salt thereof), or to amount of relevant active form of methylnaltrexone for a particular purpose (e.g., opioid antagonism), whether or not other forms of methylnaltrexone are also present. Furthermore, as indicated herein, dosages or amounts are sometimes defined with reference to a particular form of methylnaltrexone (e.g., N-methylnaltrexone bromide). Where a different form or salt of methylnaltrexone is used, those of ordinary skill in the art will appreciate that such dosages or amounts may be adjusted to a dose or amount that provides an equivalent amount of active methylnaltrexone.

Furthermore, those of ordinary skill in the art appreciate that, as with any biologically active agent, the exact amount of methylnaltrexone that is required to achieve a pharmaceutically effective amount may vary from subject to subject, depending on species, age, weight, and general condition of a subject, severity and/or incidence of the side effects or disorder, identity of the particular compound(s), mode of administration, other therapies being received and/or disorders or conditions suffered, and the like.

The exact amount of methylnaltrexone (or combination of methylnaltrexone and any other particular active agent) that is required to achieve a pharmaceutically effective amount will vary from subject to subject, depending on species, age, and general condition of a subject, severity of the side effects or disorder, identity of the particular compound(s), mode of administration, and the like. A total daily dosage of methylnaltrexone (e.g., methylnaltrexone bromide) will typically be in the range 10-200 mg, preferably 20-100 mg for a 70 kg adult human. A unit dosage formulation according to the invention will usually contain 1-250 mg of active compound (e.g., methylnaltrexone bromide) per unit, 5-100 mg of active compound per unit, 10-50 mg of active compound per unit, or about 8 mg or about 12 mg or about 16 mg or about 24 mg of active compound per unit. In certain embodiments, an effective amount of a methylnaltrexone for administration to a 70 kg adult human may comprise about 10 mg to about 50 mg of compound (e.g., methylnaltrexone bromide) per unit dosage, to be administered one or more times a day. It will be appreciated that dose ranges set out above provide guidance for the administration of active compound to an adult. The amount to be administered to for example, an infant or a baby can be determined by a medical practitioner or person skilled in the art and can be lower or the same as that administered to an adult.

In certain embodiments of the invention, an effective amount of methylnaltrexone bromide for administration to a 70 kg adult human may comprise about 10 mg to about 50 mg of compound per unit dosage, to be administered one or more times a day, an amount of methylnaltrexone equivalent to about 10-50 mg of methylnaltrexone bromide.

A once daily unit dosage preparation according to the invention will usually contain an amount of methylnaltrexone equivalent to about 1-250 mg of methylnaltrexone bromide per unit. In some embodiments, a once daily unit dosage preparation will contain an amount equivalent to about 5-100 mg of methylnaltrexone bromide per unit, or to about 10-50 mg of methylnaltrexone bromide per unit, or to about 8 mg or 12 mg or 16 mg or 24 mg of methylnaltrexone bromide per unit.

A unit dosage preparation according to the invention may contain an amount of methylnaltrexone equivalent to about 1-250 mg of methylnaltrexone bromide per unit. In some embodiments, such a unit dosage preparation may contain an amount equivalent to about 1-200 or 10-100 mg of methylnaltrexone bromide per unit, or to about 15-50 mg of methylnaltrexone bromide per unit, or to about 20-30 mg of methylnaltrexone bromide per unit. In some embodiments, inventive unit dosage preparations contain an amount of methylnaltrexone equivalent to about 10-50 mg of methylnaltrexone bromide. In certain embodiments, the present invention provides unit dosage preparations containing about 12 mg of methylnaltrexone bromide. In other embodiments, the present invention provides unit dosage preparations containing about 24 mg of methylnaltrexone bromide.

### Formulations

It has been surprisingly found that lyophilization of methylnaltrexone with a single filler or a single cryoprotectant, in the absence of additional excipients, provides a stable form of methylnaltrexone which may be stored for extended periods. Thus, the present invention demonstrates provides dry powder formulations (e.g., an amorphous powder, optionally in the form of a cake) of methylnaltrexone and a single filler or single cryoprotectant. Such dry powder formulations may be stored, then utilized for administration to a subject, when desirable, by reconstitution with a liquid. The present invention provides stable dry powder compositions, and associated methods, that deliver methylnaltrexone. In certain embodiments, provided formulations can maintain integrity without substantial production of degradants following storage, including storage under room temperature. Thus, provided formulations can confer improved storage stability of methylnaltrexone. In some embodiments, provided formulations contain reduced levels of a degradant produced by Hofmann elimination of methylnaltrexone.

In particular, the present invention provides stable formulations for administration to subjects. In some embodiments, provided formulations are useful for parenteral administration. Provided formulations and compositions, or dosage preparations comprising them, may include dry powder (e.g., lyophilized) compositions, solutions for injection, suspensions for injection, dry powder compositions for reconstitution by combination with an appropriate solvent or other medium prior to use, emulsions, dispersions, etc. In some embodiments, such formulations, compositions, and/or dosage preparations are sterile.

In some embodiments, a formulation according to the present invention is a dry material that consists essentially of methylnaltrexone and a single other agent. In some embodiments, the dry material is in cake form.

In certain embodiments, inventive dry powder formulations are amorphous. The term “amorphous” means a physical state lacking significant crystal lattice structure and may be verified by X-ray diffraction, solid-state NMR (SSNMR) and/or other supportive means known in the art, such as observation with a polarized light microscope and Differential Scanning calorimetry (DSC). In some embodiments, provided dry powder formulations are substantially free of detectable discrete crystals. Without wishing to be bound by any particular theory, Applicants note that formulations lacking discrete crystals may be particularly desirable both because they permit intimate contact between methylnaltrexone and the filler or cryoprotectant, and because they typically have consistent solubility profiles, etc. By contrast, if discrete crystals are present in a formulation, those crystals may have different stability and/or solubility characteristics than other portions of the formulation. In some embodiments, the present invention provides an amorphous dry material consisting essentially of methylnaltrexone and a single filler or single cryoprotectant.

In some embodiments, a dry powder formulation is reconstituted in appropriate liquid, so that a solution, suspension, emulsion or dispersion consisting essentially of methylnaltrexone, a single filler or single cryoprotectant, and a reconstituting liquid is generated. The present invention comprises methods of preparing and/or administering such reconstituted solutions, suspensions, emulsions, or dispersions to subject(s). Thus, provided by the present invention are methods for preparing a composition comprising a formulation consisting of essentially of methylnaltrexone and a single filler or single cryoprotectant in an appropriate liquid. In some embodiments, reconstituted preparations are further diluted with an aqueous carrier, e.g., for intravenous administration.

In some embodiments of the invention, methylnaltrexone may comprise from about 10% to about 90% of the formulation. In some embodiments, methylnaltrexone may comprise from about 5%, 10%, about 20%, about 30%, about 40%, about 50%, about 60%, 70%, 80%, or about 90% of the formulation. In some embodiments, the formulation may comprise an amount of methylnaltrexone equivalent to about 5%, 10%, 20%, 30%, 40%, 50%, 60%, 70%, 80%, or 90% methylnaltrexone bromide.

In many embodiments, provided formulations include methylnaltrexone together with a single filler or single cryoprotectant. Those of ordinary skill in the art will appreciate that any material capable of providing bulk can act as a filler. The present invention encompasses the recognition that merely providing filling/bulking capability may help stabilize composition of methylnaltrexone. In some embodiments, a particular agent may further have particular stabilizing attributes, for example due to its ability to interact with methylnaltrexone, thereby potentially affecting reactions, including degradation reactions available to the compound. Agents with such stabilizing attributes generally are often termed “preservatives” in the art. Agents with stabilizing attributes under conditions of freeze drying are often termed “cryoprotectants”.

In some embodiments, a filler or cryoprotectant may comprise from about 10%, about 20%, about 30%, about 40%, about 50%, about 60%, 70%, 80%, 90% or about 95% of the formulation, based upon total weight of the formulation. In some embodiments, a cryoprotectant may comprise from about 25%, about 35%, about 45%, about 55%, about 65% or about 75% of the formulation, based upon total weight of the formulation.

In some embodiments, filler or cryoprotectant may be present in a ratio with methylnaltrexone that is close to 1:1; in other embodiments, the filler/cryoprotectant:methylnaltrexone ratio may be within the range of about 2:1, 3:1, 4:1, 5:1 or more. In some embodiments of the invention, formulations including smaller amounts of methylnaltrexone have a higher ratio of filler or cryoprotectant to methylnaltrexone.

In some embodiments of the invention, dry powder formulations containing filler or cryoprotectant have fewer methylnaltrexone degradation products than do otherwise identical formulations, stored under comparable conditions for a comparable amount of time, that lack filler or cryoprotectant. In some embodiments of the invention, dry powder formulations containing a higher ratio of filler/cryoprotectant to methylnaltrexone have fewer methylnaltrexone degradation products than do otherwise identical formulations, stored under comparable conditions for a comparable amount of time, that contain lower ratios. In either such comparison, the phrase “fewer methylnaltrexone degradation products” can refer either to a smaller number of degradation products or to a lower amount of a particular degradation product. In some embodiments, a lower amount of a degradation product produced by Hofmann elimination of methylnaltrexone is present.

In some embodiments, provided dry powder formulations consist essentially of methylnaltrexone and a single other agent and do not contain more than 2% of methylnaltrexone degradation products. That is, in general, a stable formulation of containing methylnaltrexone does not accumulate methylnaltrexone degredation products to a level above 2% over a designated period of time. In some embodiments, no material increase (as compared with an initial amount present at production of the formulation) in degradation products is observed over a designated period of time. In some embodiments, such a stable formulation containing methylnaltrexone does not accumulate methylnaltrexone degredants to a level above 1.5%, 1.4%, 1.3%, 1.2%, 1.1%, 1.0%, 0.9%, 0.8%, 0.7%, 0.6%, 0.5%, 0.4%, 0.3%, 0.2%, 0.15%, 0.1% or less.

Any of a variety of agents may be utilized as a filler or cryoprotectant according to the present invention. For example, histidine, polyethylene glycol, polyvinyl pyrrolidine, lactose, dextran, sucrose, and/or mannitol may be utilized, in any appropriate form. In some embodiments lactose is utilized; in some embodiments, the lactose is lactose monohydrate. Without wishing to be bound by any particular theory, we note that lactose monohydrate is a reducing sugar and has certain abilities to bind with other molecules that may impart cryoprotectant characteristics.

In some embodiments of the invention, where a dry powder formulation contains a filler or cryoprotectant other than lactose monohydrate, it contains an amount or percentage that is equivalent to the recited amount or percentage of lactose monohydrate.

In some embodiments, the formulation consists essentially of methylnaltrexone (in any appropriate form), and a single filler or single cryoprotectant. In some embodiments, the formulation consists essentially of methylnaltrexone and lactose. In some embodiments lactose is lactose monohydrate. In certain embodiments the formulation consists essentially of methylnaltrexone bromide and lactose monohydrate. Thus, the present invention provides dry preparations that consist essentially of methylnaltrexone bromide and lactose monohydrate. In some embodiments such dry preparations are in the form of an amorphous cake.

In certain embodiments, a formulation consists essentially of methylnaltrexone, wherein the methylnaltrexone is present in an amount equivalent to about 2 mg to about 200 mg of methylnaltrexone bromide, and a single filler or single cryoprotectant, present in an amount equivalent to about 10 mg to about 200 mg of lactose monohydrate. In certain embodiments, is present in an amount equivalent to about 2 mg to about 100 mg of methylnaltrexone bromide, and filler or cryoprotectant is present in an amount equivalent to about 10 mg to about 100 mg of lactose monohydrate. In some embodiments, methylnaltrexone is present in an amount equivalent to about 5 mg to about 50 mg, or to about 5 mg to about 25 mg, or to about 8 mg to about 25 mg, or to about 12 mg to about 25 mg of methylnaltrexone bromide, and filler or cryoprotectant is present in an amount equivalent to about 10 mg to about 50 mg, or to about 20 mg to about 50 mg, or to about 25 mg to about 45 mg, or to about 30 mg to about 42 mg, or to about 35 mg to about 40 mg of lactose monohydrate.

In some embodiments, provided dry powder formulations consisting essentially of methylnaltrexone and a single filler or a single cryoprotectant are stable for at least one month, two months, three months, four months, five months, six months or more. In some embodiments, provided formulations are stable for 12 months or more. In some embodiments, provided formulations are stable at room temperature.

Dry powder formulations may be reconstituted with a liquid carrier so as to generate a resulting reconstitute composition. In many embodiments, the liquid carrier will be an aqueous carrier. Reconstituted compositions may thus comprise an admixture of methylnaltrexone, filler or cryoprotectant, and an appropriate liquid carrier. An appropriate liquid carrier for reconstitution of dry powder compositions may comprise an aqueous carrier such as water (e.g., sterile water or water for injection) or an isotonic solution. A reconstituted composition, for example, may be prepared, for example, to have methylnaltrexone at a concentration with a range of about 0.1 mg/mL to about 50 mg/ml, or within a range of about 0.2 mg/mL to about 48 mg/mL, or within a range of about 0.24 mg/mL to about 4.8 mg/mL. In certain embodiments, the present invention provides a reconstituted composition having methylnaltrexone at a concentration of about 5 mg/mL.

Aqueous carriers are known in the art, and include, but are not limited to sterile water, water for injection, or an isotonic solution. An isotonic solution comprises an isotonic agent solution. Pharmaceutically acceptable isotonic solutions include, but are not limited to sodium chloride solution, Ringer's injection, isotonic dextrose injection, dextrose and lactated Ringers injection. In some embodiments, provided compositions comprises water for injection. In some embodiments, the present invention provides reconstituted formulations that consist essentially of methylnaltrexone, cryoprotectant and water. In some embodiments, reconstituted formulations consist essentially of methylnaltrexone, a cryoprotectant, and an isotonic solution.

An isotonic agent useful according to the present invention can be any pharmaceutically acceptable isotonic agent, or a solution thereof. Common isotonic agents include agents selected from the group consisting of sodium chloride, mannitol, lactose, dextrose (hydrous or anhydrous), sucrose, glycerol, and sorbitol, or a solution of any of the foregoing. In certain embodiments, a provided reconstituted formulation comprises an isotonic agent which is sodium chloride or a solution thereof. In some embodiments, sodium chloride is present in an isotonic amount, such that final concentration of sodium chloride is about 0.1%, about 0.25%, about 0.65% or about 0.9%.

In some embodiments, a provided reconstituted formulation consists essentially of methylnaltrexone, lactose, and an isotonic solution. In some embodiments, a provided reconstituted formulation consists essentially of methylnaltrexone, lactose, water for injection, and sodium chloride in an amount such that the final concentration is isotonic sodium chloride (e.g., 0.9% 0.65%, 0.25%, 0.1% sodium chloride). In any such embodiments, methylnaltrexone may comprise methylnaltrexone bromide, and the lactose may comprise lactose monohydrate.

### Dosage, Administration, and Dosage Preparations

Dry powder formulations may be prepared, and/or may be reconstituted, for administration to subject(s). For example, dry powder formulations may be prepared and/or reconstituted for parenteral administration.

Parenteral administration of a composition comprising a reconstituted formulation may include any of intravenous injection, intravenous infusion, intradermal, intralesional, intramuscular, subcutaneous injection or depot administration of a unit dosage. A unit dosage may or may not constitute a single “dose” of active compound(s), as a prescribing doctor may choose to administer more than one, less than one, or precisely one unit dosage in each dose (i.e., each instance of administration). For example, unit dosage(s) may be administered once, less than once, or more than once a day, for example, once a week, once every other day, once a day, or 2, 3 or 4 times a day, usually 1 to 3 times a day, more preferably 1 or 2 times per day. In some embodiments, particularly where a unit dosage is to be delivered intravenously, it is delivered by periodic infusion several times a day over a series of days (that may be continuous or interrupted). In some embodiments, intravenous formulations are delivered by periodic infusions spaced apart by several (e.g., about 2-10) hours over several (e.g., about 2-20, about 4-15, about 6-12, about 10) days. In some embodiments, intravenous formulations are delivered over consecutive days. As will be appreciated by those of ordinary skill in the art, the administration regimen may be adjusted, for example, according to the characteristics of the individual receiving treatment and/or of the precise situation (e.g., treatment of side effect associated with chronic opioid therapy, associated with acute opioid exposure, and/or associated with activity of endogenous opioids, etc.). To give but one example, a shorter administration regimen may be appropriate for rescue applications, whereas other applications may involve correlating methylnaltrexone therapy with term or timing of opioid exposure or activity.

The present invention provides variety of different dosage preparations useful for parenteral administration, including, for example, formulations provided in a container (e.g., a vial, ampoule, syringe, bag, dispenser, etc.). In some embodiments, a formulation is provided in a vial or syringe. In some embodiments, a formulation is provided in a vial or syringe containing a unit dosage of methylnaltrexone. In such embodiments, a formulation may comprise about 1 mg to about 200 mg methylnaltrexone bromide. In some embodiments, the unit dosage contains from about 1 mg to about 80 mg, from about 5 mg to about 50 mg, or from about 7.5 mg to about 40 mg. In some embodiments, the unit dosage contains about 8 mg, about 12 mg, about 16 mg, or about 24 mg methylnaltrexone; if such methylnaltrexone is not in the form of methylnaltrexone bromide, then it may be present in an amount equivalent to the recited amount of methylnaltrexone bromide.

In one embodiment, a formulation is provided in a vial containing dry powder that consists essentially of methylnaltrexone, and a filler or cryoprotectant. In one embodiment, a formulation is provided in a syringe containing dry powder that consists essentially of methylnaltrexone, and a filler or cryoprotectant.

In one embodiment, provided is a vial containing a dry powder formulation consisting essentially of methylnaltrexone and a filler or cryoprotectant, and sufficient room to allow addition of an appropriate solvent for reconstitution of the dry powder formulation. In one embodiment, a composition can be prepared by adding to an appropriate liquid (e.g., solvent) a dry powder formulation consisting essentially of methylnaltrexone and lactose (e.g., lactose monohydrate).

In one embodiment, provided is a syringe or dispenser containing a dry powder formulation that consists essentially of methylnaltrexone and a filler or cryoprotectant, and sufficient room to allow addition of an appropriate solvent or liquid for reconstitution. In one embodiment, a formulation in a syringe or dispenser is prepared with reconstituted methylnaltrexone formulation, where the solution consists essentially of methylnaltrexone, lactose (in an appropriate form, e.g., lactose monohydrate), and an appropriate liquid carrier. In one embodiment, a composition can be prepared comprising a dry powder formulation consisting essentially of methylnaltrexone, and a filler or cryoprotectant, in an isotonic solution.

In certain embodiments, dosage preparations are provided that allow reconstitution of a dry powder formulation as a dose concentrate. A dose concentrate may be used over a standard treatment interval such as immediately upon reconstitution or up to about 24 hours after reconstitution, as appropriate. In certain embodiments, a dose-concentrate is prepared by reconstituting a dry powder formulation in a container (e.g., glass or plastic bottle, vial, ampoule, etc.) in a sufficient amount to treat a subject for a period ranging from 6 hours to 1 week, but preferably from 12 hours to 24 hours. A suitable container may desirably have an empty space of sufficient size to permit (i) addition of liquid carrier plus (ii) additional space as necessary to permit agitation and effect complete solution or suspension of the dry powder composition in the added liquid carrier. A container may be equipped with a penetrable top, for example, a rubber seal, so that the liquid carrier may be added (and/or reconstituted composition may be removed) by penetrating the seal with a hypodermic syringe. In some embodiments, a needle-less penetrable seal is utilized.

An example of a dosage preparation useful for preparation of a unit dose or a dose concentrate can include a vial having a capacity of from about 1 mL to about 100 mL, or any appropriate capacity in between (e.g., 5 mL, 10 mL, 20 mL, 25 mL, 50 mL, 75 mL, etc.) In some embodiments, a vial with a capacity from about 1 mL to about 100 mL may contain about 1 mg to about 4 g of dry powder formulation. In some embodiments, a 10 mL glass vial is utilized, containing about 5 mg to about 400 mg of methylnaltrexone. In some embodiments, a 10 mL glass vial contains about 5 mg to about 200 mg, or about 5 mg to about 100 mg, or about 10 mg to about 75 mg, or about 25 mg of methylnaltrexone. If the methylnaltrexone is not in the form of methylnaltrexone bromide, an amount equivalent to the recited amount of methylnaltrexone bromide may be present.

In certain embodiments, a 10 mL glass vial contains about 8 mg of methylnaltrexone, about 12 mg of methylnaltrexone, or about 24 mg of methylnaltrexone. If the methylnaltrexone is not in the form of methylnaltrexone bromide, an amount equivalent to the recited amount of methylnaltrexone bromide may be present.

In some embodiments, a 10 mL glass vial contains about 5 mg to about 200 mg of dry powder formulation, about 5 mg to about 100 mg of dry powder formulation, about 10 mg to about 75 mg of dry powder formulation, or about 50 mg of dry powder formulation.

A non-limiting specific example of a provided dosage preparation is a 10 mL glass vial with a rubber seal having a 1 dry powder formulation containing methylnaltrexone and a filler or cryoprotectant such as lactose (e.g., lactose monohydrate). In some embodiments, empty space is present around the solid composition contents of the container, allowing ample room for addition of a liquid carrier such as a solvent or diluent (e.g., sterile water for injection, isotonic solution (e.g., saline)), plus additional room sufficient to allow for agitation of contents.

Addition of liquid carrier to a dry powder formulation can be used to prepare a unit dose or a dose concentrate which may then be conveniently used to form unit dosages of liquid pharmaceutical formulations by removing aliquot portions or entire contents for further dilution. Reconstituted dose concentrate can be added, for example, to an intravenous (IV) container containing a suitable aqueous carrier for administration to a subject. Useful aqueous carriers include standard solutions for injection as previously described (e.g., 5% dextrose, saline, or sterile water etc.). Typical unit dosage IV bags are conventional glass or plastic containers having inlet and outlet means and having standard (e.g., 50 mL, 100 mL and 150 mL) capacities. A dose concentrate solution can be added to a unit dosage IV bag in an amount sufficient to achieve a concentration of about 0.1 mg/mL to about 1.0 mg/mL of methylnaltrexone, or about 0.24 mg/mL to about 0.48 mg/mL in the unit dosage IV bag.

In one embodiment, a provided formulation is in a syringe or other dispenser filled with a provided formulation as described above and herein. In some embodiments, a syringe or dispenser has a capacity from about 1 mL to about 20 mL. In some embodiments a syringe or dispenser has a capacity of about 1 mL, about 2 mL, about 2.5 mL, about 5 mL, about 7.5 mL, about 10 mL, about 15 mL, or about 20 mL. In some embodiments, a syringe or dispenser utilizes a hypodermic needle for administration of contents of the syringe or dispenser to a subject. In certain embodiments, a syringe or dispenser utilized a needle-less adapter for transfer of contents of the container to a subject, or, alternatively to a second container for mixing and/or dilution of contents with another solution.

A container may be equipped with a penetrable or spikable top, for example, a rubber seal, such that aqueous solvent may be added by penetrating the seal with a hypodermic syringe or other type non-needle based, penetrable seal in order to transfer concentrate contents. In certain embodiments, a provided formulation is provided in a spikable vial. In some embodiments, a provided formulation is provided in a 10 mL spikable vial.

Addition of aqueous solvent to a liquid dose concentrate may be conveniently used to form unit dosages of liquid pharmaceutical formulations by removing aliquot portions or entire contents of a dose concentrate for dilution. Dose concentrate may be added to an intravenous (IV) container containing a suitable aqueous solvent. Useful solvents are standard solutions for injection as previously described (e.g., 5% dextrose, saline, lactated ringer's, or sterile water for injection, etc.). Typical unit dosage IV bags are conventional glass or plastic containers having inlet and outlet means and having standard (e.g., 25 mL, 50 mL, 100 mL and 150 mL) capacities. Dose concentrate solution of a pharmaceutical formulation of the invention is added to a unit dosage IV container in an amount to achieve a concentration of about 0.1 to about 1.0 mg of methylnaltrexone per mL and preferably from about 0.24 to about 0.48 mg per mL.

In other embodiments, it may be desirable to package a provided dosage form in a container to protect the formulation from light until usage. In some embodiments, use of such a light-protective container may inhibit one or more degradation pathways. For example, a vial may be a light container which protects contents from being exposed to light. Additionally and/or alternatively, a vial may be packaged in any type of container which protects a formulation from being exposed to light (e.g., secondary packaging of a vial). Similarly, any other type of container may be a light protective container, or packaged within a light protective container.

### Preparation of Dry Powder Formulations

Dry powder formulations of the present invention may be prepared in accordance with any of a variety of known techniques, for example as described by M. E. Aulton in “Pharmaceutics: The Science of Dosage Form Design” (1988) (Churchill Livingstone), the relevant disclosures of which are hereby incorporated by reference.

Dry powder formulations may be prepared by conventional lyophilization methods or by other techniques such as spray drying, or blending of dry powders of the appropriate salts of the individual or combined ingredients. Lyophilization methods can include tray lyophilization and vial lyophilization. Vial lyophilization methods may be advantageous for preparing multiple dosage preparations, each containing a unit dosage of methylnaltrexone.

In certain embodiments, lyophilized formulations, are prepared by first providing a solution or suspension of methylnaltrexone and/or appropriate filler or cryoprotectant in an appropriate solvent. If desired, prepared methylnaltrexone solution or suspension may be subjected to a filtration process before lyophilization. Such a filtration process may include, for example, a sterilizing filtration and/or an ultra filtration of the processing solution before lyophilization to eliminate microorganisms or other contaminating matter from the processing solution before lyophilization.

If desired, methylnaltrexone solution or suspension may be subjected to a distributing process before lyophilization. A distributing process may include, for example in the case of vial lyophilizations, distributing a suitable volume of the processing solution before lyophilization into vials, taking the concentration of methylnaltrexone into consideration in order that vial products carry a desired amount of methylnaltrexone.

In some embodiments, lyophilization of the composition is performed by a controlled freeze-drying process. For example, a methylnaltrexone solution can be subjected to a temperature treating process (e.g., to improve cake characteristics), and then can be dried in a high vacuum for sublimating liquid carrier. For example, a solution may first be frozen, then subjected to a low pressure environment (e.g., vacuum) to facilitate sublimation, and then gently heated to optimize drying rate of the product.

Any available technique can be employed to obtain a liquid solution or suspension containing methylnaltrexone and filler or cryoprotectant suitable for lyophilization. For example, a solution or suspension of methylnaltrexone may be prepared or obtained to which filler/cryoprotectant is added; a solution or suspension of filler/cryoprotectant may be prepared or obtained to which methylnaltrexone is added, or both methylnaltrexone and filler/cryoprotectant can be added to a liquid carrier (e.g., simultaneously or sequentially, including in interdigitated amounts).

To give but one example, methylnaltrexone (in any appropriate form, e.g., methylnaltrexone bromide, etc.) can be dissolved or suspended in a suitable amount of liquid carrier (e.g., water, isotonic saline), and optionally mixed. A suitable filler or cryoprotectant (e.g., lactose, for example in the form of lactose monohydrate) is added and optionally mixed. In some embodiments, a liquid carrier may be an aqueous solvent such as water, purified water, water for injection, or isotonic sodium chloride solution. In some embodiments, the liquid carrier is water for injection.

A typical process for preparing a lyophilized composition comprises sequential steps of: (a) preparing or obtaining a solution or suspension consisting essentially of methylnaltrexone, an aqueous solvent and a filler or cryoprotectant, (b) freezing the composition to a temperature of from about −10° C. to about −75° C., wherein the temperature is maintained for at least about 30 minutes to about 5 hours, (c) applying a vacuum for at least about 5-30 minutes during or after freezing; (d) carrying out a primary drying by changing the temperature to a primary drying temperature from about −30° C. to about 30° C., and maintaining the temperature at the primary drying temperature for at least about 10-40 hours to produce a primary lyophilate, and (e) carrying out a secondary drying comprising raising the temperature to a secondary drying temperature from about 0° C. to about 60° C., and maintaining the temperature at the secondary drying temperature for at least about 5 hours, or until the lyophilate reaches a particular temperature, resulting in production of a lyophilized formulation consisting essentially of methylnaltrexone and a filler or cryoprotectant.

One particular process may comprise the sequential steps of: (a) dissolving lyophilized composition ingredients comprising: methylnaltrexone bromide and a single filler or single cryoprotectant (e.g., lactose (e.g., lactose monohydrate) in an aqueous solvent (e.g., water for injection); (b) cooling the solution of step (a) to a temperature below −35° C. and maintaining the solution below −35° C. for a period; (c) evacuating the lyophilizer to a pressure of about 300 uM Hg (40 pascals) or less, and maintaining such reduced pressure for an additional period of at most about 10-30 minutes; (d) heating the product in the lyophilizer on a shelf set to about +20° C.; (e) maintaining these conditions, under subatmospheric pressure for a time sufficient (e.g., about 10-15 hours) to yield a solid lyophilized product; (f) drying at about +35° C. Preferably, step (b) is performed for a time period of at least 2 hours, and step (e) is preferably conducted for a period of at least 14 hours and step (f) is performed at a subatmospheric pressure less than about 100 mTorr (40 pascals) and conditions are maintained for 5 hours after the shelf temperature at +40° C. has been achieved, or until the product temperature is above 30° C.

Provided methylnaltrexone compositions may be subjected to a distributing process to vials (e.g., clear glass vial, amber vials), ampoules, syringes, or dispensers (e.g., auto-dispensers) before or after lyophilization. Such a distributing process may include, for example in the case of vial packaging, a process distributing a suitable amount of dry powder composition into vials, taking the concentration or amount of methylnaltrexone into consideration in order that vial products carry a desired amount of methylnaltrexone.

In one embodiment, dry powder compositions are incorporated into vials, ampoules, syringes, or dispensers, either before or after lyophilization or other drying process, as described herein. Various packaging systems may optionally be utilized in conjunction with provided compositions.

### Combination Products and Combined Administration

In some embodiments, provided formulations may optionally be used in combination or in conjunction with compositions comprising at least one other active compound. In some embodiments, provided formulations include one or more other active compounds in addition to methylnaltrexone. In such combination formulations, additional compound(s) may be included in one or more portion(s) that include methylnaltrexone, may be missing from one or more portions that include methylnaltrexone, and/or may be included in one or more portions that do not include methylnaltrexone. Some embodiments of the invention therefore provide formulations that deliver at least methylnaltrexone and at least one other active compound. Additionally, the invention encompasses formulations that deliver at least two independent portions of methylnaltrexone, and that further deliver at least one other active compound(s).

For example, a reconstituted dose concentrate provided herein may be further diluted in a carrier suitable for IV administration in conjunction or in combination with a composition for IV administration which comprises an opioid and/or opioid antagonist. Such combination products containing both an opioid and an opioid antagonist would allow simultaneous relief of pain and minimization of opioid-associated side effects (e.g., gastrointestinal effects (e.g., delayed gastric emptying, altered GI tract motility), etc.).

Opioids useful in analgesic applications are known in the art. For example, opioid compounds include, but are not limited to, alfentanil, anileridine, asimadoline, bremazocine, burprenorphine, butorphanol, codeine, dezocine, diacetylmorphine (heroin), dihydrocodeine, diphenoxylate, ethylmorphine, fedotozine, fentanyl, funaltrexamine, hydrocodone, hydromorphone, levallorphan, levomethadyl acetate, levorphanol, loperamide, meperidine (pethidine), methadone, morphine, morphine-6-glucoronide, nalbuphine, nalorphine, nicomorphine, opium, oxycodone, oxymorphone, papavereturn, pentazocine, propiram, propoxyphene, remifentanyl, sufentanil, tilidine, trimebutine, and tramadol. In some embodiments the opioid is at least one opioid selected from alfentanil, buprenorphine, butorphanol, codeine, dezocine, dihydrocodeine, fentanyl, hydrocodone, hydromorphone, levorphanol, meperidine (pethidine), methadone, morphine, nalbuphine, nicomorphine, oxycodone, oxymorphone, papavereturn, pentazocine, propiram, propoxyphene, sufentanil and/or tramadol. In certain embodiments, the opioid is selected from morphine, codeine, oxycodone, hydrocodone, dihydrocodeine, propoxyphene, fentanyl, tramadol, and mixtures thereof. In a particular embodiment, the opioid is loperamide. In another particular embodiment, the opioid is hydromorphone. In other embodiments, the opioid is a mixed agonist such as butorphanol. In some embodiments, the subjects are administered more than one opioid, for example, morphine and heroin or methadone and heroin.

The amount of additional active compound(s) present in combination compositions or used in conjunction with compositions of this invention will typically be no more than the amount that would normally be administered in a composition comprising that active compound as the only therapeutic agent. In certain embodiments, the amount of additional active compound will range from about 50% to 100% of the amount normally present in a composition comprising that compound as the only therapeutic agent.

In certain embodiments, provided formulations may also be used in conjunction with and/or in combination with conventional therapies for gastrointestinal dysfunction to aid in the amelioration of constipation and bowel dysfunction, For example, conventional therapies include, but may not be limited to functional stimulation of the intestinal tract, stool softening agents, laxatives (e.g., diphelymethane laxatives, cathartic laxatives, osmotic laxatives, saline laxatives, etc), bulk forming agents and laxatives, lubricants, intravenous hydration, and nasogastric decompression.

### Kits and Uses of Inventive Formulations

Uses

As discussed above, the present invention provides methods and formulations useful in antagonizing undesirable side effects of opioid activity, including of opioid analgesic therapy (e.g., gastrointestinal effects (e.g., delayed gastric emptying, altered GI tract motility), etc.). In certain embodiments, formulations of the invention may be used to treat subjects having disease states that are ameliorated by any treatment where temporary suppression of the μ opioid receptor system is desired (e.g., ileus, etc.). In certain embodiments, provided formulations are used in human subjects.

Accordingly, administration of provided formulations may be advantageous for treatment, prevention, amelioration, delay or reduction of side effects of opioid administration, such as, for example, gastrointestinal dysfunction (e.g., inhibition of intestinal mobility, constipation, GI sphincter constriction, nausea, emesis (vomiting), biliary spasm, opioid bowel dysfunction, colic) dysphoria, pruritis, urinary retention, depression of respiration, papillary constriction, cardiovascular effects, chest wall rigidity and cough suppression, depression of stress response, and immune suppression associated with use of narcotic analgesia, etc, or combinations thereof. Use of provided formulations may thus be beneficial from a quality of life standpoint for subjects receiving administration of opioids, as well as to reduce complications arising from chronic constipation, such as hemorrhoids, appetite suppression, mucosal breakdown, sepsis, colon cancer risk, and myocardial infarction.

In some embodiments, provided formulations are useful for administration to a subject receiving short term opioid administration. In some embodiments, provided formulations are useful for administration to patients suffering from post-operative gastrointestinal dysfunction.

In other embodiments, provided formulations are useful for administration to subjects receiving chronic opioid administration (e.g., terminally ill patients receiving opioid therapy such as an AIDS patient, a cancer patient, a cardiovascular patient; subjects receiving chronic opioid therapy for pain management; subjects receiving opioid therapy for maintenance of opioid withdrawal). In some embodiments, the subject is a subject using opioid for chronic pain management. In some embodiments, the subject is a terminally ill patient. In other embodiments the subject is a person receiving opioid withdrawal maintenance therapy.

Additional uses for formulations described herein may be to treat, reduce, inhibit, or prevent effects of opioid administration including, e.g., aberrant migration or proliferation of endothelial cells (e.g., vascular endothelial cells), increased angiogenesis, and increase in lethal factor production from opportunistic infectious agents (e.g., Pseudomonas aeruginosa). Additional advantageous uses of provided formulations include treatment of opioid-induced immune suppression, inhibition of angiogenesis, inhibition of vascular proliferation, treatment of pain, treatment of inflammatory conditions such as inflammatory bowel syndrome, treatment of infectious diseases and diseases of the musculokeletal system such as osteoporosis, arthritis, osteitis, periostitis, myopathies, and treatment of autoimmune diseases.

In certain embodiments, formulations of the invention may be used in methods for preventing, inhibiting, reducing, delaying, diminishing or treating gastrointestinal dysfunction, including, but not limited to, irritable bowel syndrome, opioid-induced bowel dysfunction, colitis, post-operative or postpartum ileus, paralytic ileus, nausea and/or vomiting, decreased gastric motility and emptying, inhibition of the stomach, and small and/or large intestinal propulsion, increased amplitude of non-propulsive segmental contractions, constriction of sphincter of Oddi, increased anal sphincter tone, impaired reflex relaxation with rectal distention, diminished gastric, biliary, pancreatic or intestinal secretions, increased absorption of water from bowel contents, gastro-esophageal reflux, gastroparesis, cramping, bloating, abdominal or epigastric pain and discomfort, constipation, idiopathic constipation, post-operative gastrointestinal dysfunction following abdominal surgery (e.g., colectomy (e.g., right hemicolectomy, left hemicolectomy, transverse hemicolectomy, colectomy takedown, low anterior resection) or hernia repair), and delayed absorption of orally administered medications or nutritive substances.

Provided formulations are also useful in treatment of conditions including cancers involving angiogenesis, immune suppression, sickle cell anemia, vascular wounds, and retinopathy, treatment of inflammation associated disorders (e.g., irritable bowel syndrome), immune suppression, chronic inflammation.

In still further embodiments, veterinary applications (e.g., treatment of domestic animals, e.g. horse, dogs, cats, etc.) of use of formulations are provided. Thus, use of provided formulations in veterinary applications analogous to those discussed above for human subjects is contemplated. For example, inhibition of equine gastrointestinal motility, such as colic and constipation, may be fatal to a horse. Resulting pain suffered by the horse with colic can result in a death-inducing shock, while a long-term case of constipation may also cause a horse's death. Treatment of equines with peripheral opioid antagonists has been described, e.g., in U.S. Patent Publication No. 20050124657 published Jan. 20, 2005.

It will also be appreciated that formulations of the present invention can be employed in combination therapies, that is, methylnaltrexone compositions can be administered concurrently with, prior to, or subsequent to, one or more other desired therapeutics or medical procedures. Particular combination therapies (therapeutics or procedures) to employ in a combination regimen will take into account compatibility of the desired therapeutics and/or procedures and the desired therapeutic effect to be achieved. It will also be appreciated that therapies employed may achieve a desired effect for the same disorder (for example, a formulation may be administered concurrently with another compound used to treat the same disorder), or they may achieve different effects (e.g., control of any adverse effects). As used herein, additional therapeutic compounds which are normally administered to treat or prevent a particular disease, or condition, are known as “appropriate for the disease, or condition, being treated”.

In other embodiments, provided formulations, as well as compositions and products comprising the provided formulations, are useful in preparation of medicaments, including, but not limited to medicaments useful in the treatment of side effects of opioid administration (e.g., gastrointestinal side effects (e.g., inhibition of intestinal motility, GI sphincter constriction, constipation, nausea, emesis), dysphoria, pruritis, etc.) or a combination thereof. Provided formulations are useful for preparations of medicaments, useful in treatment of patients receiving short term opioid therapy (e.g., patients suffering from post-operative gastrointestinal dysfunction receiving short term opioid administration) or subjects using opioids chronically (e.g., terminally ill patients receiving opioid therapy such as an AIDS patient, a cancer patient, a cardiovascular patient; subjects receiving chronic opioid therapy for pain management; or subjects receiving opioid therapy for maintenance of opioid withdrawal). Still further, preparation of medicaments useful in the treatment of pain, treatment of inflammatory conditions such as inflammatory bowel syndrome, treatment of infectious diseases, treatment of diseases of the musculoskeletal system such as osteoporosis, arthritis, osteitis, periostitis, myopathies, treatment of autoimmune diseases and immune suppression, therapy of post-operative gastrointestinal dysfunction following abdominal surgery (e.g., colectomy (e.g., right hemicolectomy, left hemicolectomy, transverse hemicolectomy, colectomy takedown, low anterior resection) or hernia repair), idiopathic constipation, and ileus, and treatment of disorders such as cancers involving angiogenesis, chronic inflammation and/or chronic pain, sickle cell anemia, vascular wounds, and retinopathy. For example, as described herein, dry powder formulations may be reconstituted with appropriate solvent. Reconstitute may be utilized as prepared as a medicament for treatment of the foregoing disorders. Additionally or alternatively, reconstitute may be further diluted for preparation of a medicament useful for treatment of the foregoing disorders.

### Pharmaceutical Kits and Packaging

Still further encompassed by the invention are pharmaceutical packs and/or kits. Pharmaceutical packs and/or kits provided may comprise a formulation and a container (e.g., a vial, ampoule, bottle, syringe, and/or dispenser package, or other suitable container). In some embodiments, contents of provided formulation in a container combine to form a unit dosage. In some embodiments, contents of provided formulation in a container can be reconstituted in a solvent to form a dose concentrate.

In some embodiments, provided kits may optionally further include a second container comprising appropriate solvent or diluent, and/or instructions for use of appropriate solvent or diluent for preparation of reconstituted formulation. In some embodiments, contents of provided formulation in a first container and solvent in a second container combine to form a unit dosage. In some embodiments, contents of provided formulation in a container and solvent in a second container combine to form a dose concentrate. In some embodiments, contents of provided formulation, container and solvent container combine to form a unit dosage. In some embodiments, contents of provided formulation container and solvent container combine to form a dose concentrate.

In still other embodiments, a third container comprising a suitable aqueous carrier for further dilution of a reconstitute for preparation of administration to a subject via IV administration.

In some embodiments, a reconstituted formulation of the invention may be useful in conjunction with patient controlled analgesia (PCA) devices, wherein a patient can administer opioid analgesia as required for pain management. In such instances, co-administration of reconstituted formulations may be useful to prevent adverse side effects of opioid administration. Thus, kits of the invention may comprise a formulation for administration of methylnaltrexone contained within a cartridge suitable for reconstitution and for use in conjunction with PCA device.

Optionally, a single container may comprise one or more compartments for containing a dry powder formulation, appropriate liquid carrier for reconstitution, and/or appropriate aqueous carrier for dilution. In some embodiments, a single container may be appropriate for modification such that the container may receive a physical modification so as to allow combination of compartments and/or components of individual compartments. For example, a foil or plastic bag may comprise two or more compartments separated by a perforated seal which may be broken so as to allow combination of contents of two individual compartments once the signal to break the seal is generated. A pharmaceutical pack or kit may thus comprise such multi-compartment containers including dry powder formulation and appropriate solvent for reconstitution and/or appropriate aqueous carrier for dilution of reconstitute. Optionally, instructions for use are additionally provided in such kits.

In some embodiments, a pharmaceutical kit comprises a dry powder formulation in a reconstitution package or container wherein a needle-less exchange mechanism allows for combination of lyophilate and aqueous carrier for dilution and/or with isotonic diluent for preparation for intravenous administration. For example, in certain non-limiting examples, a dry powder formulation of the invention may be utilized in conjunction with a MINIBAG® Plus reconstitution package system (Baxter), or an ADD VANTAGE® reconstitution package (Hospira) system.

Optionally, instructions for use are additionally provided in such kits of the invention. Such instructions may provide, generally, for example, instructions for dosage and administration. In other embodiments, instructions may further provide additional detail relating to specialized instructions for particular containers and/or systems for administration. Still further, instructions may provide specialized instructions for use in conjunction and/or in combination with additional therapy. In one non-limiting example, the formulations of the invention may be used in conjunction with opioid analgesia administration, which may, optionally, comprise use of a patient controlled analgesia device (PCA). Thus, instructions for use of provided formulations may comprise instructions for use in conjunction with PCA administration devices.

In order that the invention described herein may be more fully understood, the following examples are set forth. It should be understood that these examples are for illustrative purposes only and are not to be construed as limiting this invention in any manner.

### EXEMPLIFICATION

### Example 1

### Preparation of a Lyophilized Methylnaltrexone Formulation

We have found while an aqueous solution of methylnaltrexone is not stable when maintained at room temperature for extended periods, a lyophilized amorphous solid cake containing methylnaltrexone and a single filler or single cryoprotectant (e.g., lactose monohydrate) is room temperature stable. For example, such lyophilized compositions may be prepared using the following components:

| Active | Methylnaltrexone bromide | (2-200 | mg) |
|---|---|---|---|
| Filler | Lactose Monohydrate | (10-200 | mg) |
| Solvent | Water | qs | |
| Oxygen minimizer | Nitrogen NF | | |
| Container | vial (e.g., Type I, flint glass, | 5-20 | mL |
| | with a 20 mm neck Spike-able | | |
| | 20 mm Lyo-stopper.) | | |

All equipment and equipment change parts were washed and sterilized prior to initiation of preparation. Clean, sterile depyrogenated vials and clean, sterile rubber stoppers were used during manufacture.

Formulations may be prepared with various amounts of methylnaltrexone and filler. For example, three formulations and corresponding amounts of reagents for preparation are shown in Table 1. For a 10 mL vial, 8.4, 12.6 or 25.2 mgs of methylnaltrexone bromide was dissolved in sterile water for injection; and 42.0, 37.8 or 25.2 mgs of lactose monohydrate dissolved in the methylnaltrexone solution, to a total volume of 2.625 mL. In the particular studies described and formulations prepared in these examples, R—N-methylnaltrexone was used having less than 0.15 weight percent S—N-methylnaltrexone based on the total weight of methylnaltrexone; other stereoisomers, or mixtures thereof, could alternatively be employed.

Solutions were prepared, filter sterilized using 0.45 μm and 0.22 μm filters, and resulting sterilized solution filled under low oxygen conditions into containers for lyophilization. Any suitable vial, ampoule, syringe or auto-dispenser may be utilized for filling in advance of lyophilization.

TABLE 1
Lyophilized Formulation

| INGREDIENTS | 8 mg/VIAL^{A B} | | 12 mg/VIAL^{A B} | | 24 mg/VIAL^{A B} | |
|---|---|---|---|---|---|---|
| Methylnaltrexone bromide | 8.4 | mg | 12.6 | mg | 25.2 | mg |
| Lactose monohydrate | 42.0 | mg | 37.8 | mg | 25.2 | mg |
| Water for Injection, USPB^{C} | qs to 2.625 | mL | qs to 2.625 | mL | qs to 2.625 | mL |

Nitrogen NF

For lyophilization of mixture: shelf temperature was set to 20° C. or to 25° C., then vials were loaded into the lyophilizer, and the shelf temperature was lowered to −45° C. or below at 1° C./min, and was held for at least 2 hours. A vacuum of at least 100 mtorr was applied for freeze-drying, then the shelf temperature was held at −45° C. for an additional 20 minutes. Primary drying was initiated by raising the shelf temperature to +5° C. or +20° C. at 0.5° C./minute, and maintaining for at least 14-17 hours.

Shelf temperature was next raised to +35° C. or to +40° C. at 0.5° C./minute for a secondary (terminal) drying, and was maintained for at least 5 hr or until the product temperature was above 30° C. The product was cooled to 25° C. at 0.5° C./minute, then the product chamber vacuum was released with 0.22 μm filtered nitrogen to ½ atm or 500 mBar (7.5 PSI).

Lyophilized formulation was packaged under nitrogen at V2 atm in a 10 mL vial with a 20 mm neck. Resulting lyophilized formulations may be stored at room temperature. Specifically, such formulations can be stored at 25° C. or below, and can tolerate excursions to 30° C.

Stoppers utilized were WPS V10-F597W 4432/50 B2TR Westar RS stoppers, which allow for needle-less transfer of reconstituted methylnaltrexone to a final reconstitution container for further dilution in preparation for administration to a subject. The needle-less function of this reconstitution container for preparation of intravenous solution aids the end user by not having to use a needle syringe to transfer the contents of the vial to a standard intravenous bag.

Vials were often protected from light, and were not frozen.

When desired for administration, lyophilized cake can be reconstituted with 10 mL of appropriate solvent such as, for example, Water for Injection USP. Solvent may typically be supplied with lyophilized methylnaltrexone, in a separated container (e.g., vial). Dissolution is achieved by addition of solvent and gentle agitation of the vial, resulting in a final drug concentration of 0.8, 1.2, or 2.4 mg/mL, as appropriate for each concentration. Following dissolution of the lyophilized cake, resulting solution is then diluted to a final methylnaltrexone bromide concentration of 0.04 mg/mL, 0.24 mg/mL, or 0.48 mg/mL by addition of 50 mL of an appropriate isotonic solution for intravenous delivery to a subject. Packaged formulations can be used to transfer dose concentrate to any appropriate intravenous container comprising appropriate diluent solution. In certain embodiments, dose concentrate is further diluted by addition of the reconstitute to a Minibag™ Plus Reconstitution Container (Baxter) for intravenous administration.

### Example 2

### Stability of a Lyophilized Methylnaltrexone Formulation

We determined the stability of lyophilized formulations by assessment of the presence of various degradant formation in the sample following a period of days of storage under specified conditions using HPLC analysis of samples following storage conditions under dark conditions in variable temperature/humidity as well as under variable light conditions. Stability studies were performed using standard pharmaceutical stability studies carried out according to ICH guidelines.

Specifically, as discussed in that patent application, at least three previously known degradation products of methylnaltrexone were demonstrated from HPLC analysis in 20 mg/mL isotonic saline solution (identified as RRT peaks at about 0.72, 0.89, and 1.48 when products were analyzed by HPLC). See, e.g., US Patent Application Publication No. 20040266806A1, published Dec. 30, 2004. We examined 20 mg/mL saline methylnaltrexone solutions for production of degradants, and identification of degradants, as well as identification of inhibitors of formation of different degradant products. We have identified and characterized degradants which accumulate in certain methylnaltrexone solutions. In these degradation experiments, and in the formulations prepared in the examples, R—N-methylnaltrexone was used having less than 0.15 weight percent S—N-methylnaltrexone based on the total weight of methylnaltrexone.

For HPLC analysis a Prodigy ODS-3 15 cm×2.0 mm, 3 μm particles (Phenomenex) HPLC column at a flow rate of 0.25 mL/min, using a water/methanol gradient was used. The following specifications were utilized for HPLC column:

Mobile Phase: Strength (Isocratic: 75:25 (v/v) 0.1% TFA in Water/Methanol
Purity: (Gradient):

Mobile Phase A=95:5 (v/v) 0.1% TFA in Water/Methanol

Mobile Phase B=35:65 (v/v) 0.1% TFA in Water/Methanol

Gradient Program:

| Time | |
|---|---|
| (Min) | % Mobile Phase A |
| 0 | 100 |
| 45 | 50 |
| 45.1 | 100 |
| 60 | 100 |

Column Temperature: 50° C.

Flow: 0.25 mL/minute

Detection: UV, 280 nm

Injection: Strength: 5 μL
Purity: 20 μL

Sample Solvent: 0.05M Dibasic Sodium Phosphate pH 6.8

The following compounds were identified in the stability studies using HPLC analysis of samples under the indicated storage conditions, and had the following associated calculated relative retention times:

| | |
|---|---|
| Methylnaltrexone bromide | RRT 1.00 |
| 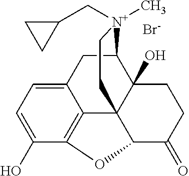 [SMILES: C[N+]1(CC2CC2)CC[C@]23c4c5ccc(O)c4O[C@H]2C(=O)CC[C@@]3(O)[C@H]1C5 ; [Br-]] | |
| Naltrexone base | RRT 1.17 |
| 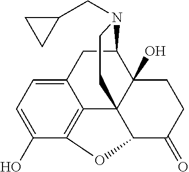 [SMILES: O=C1CC[C@@]2(O)[C@H]3Cc4ccc(O)c5c4[C@@]2(CCN3CC2CC2)[C@H]1O5] | |
| S-Methylnaltrexone bromide | RRT 0.89 |
| 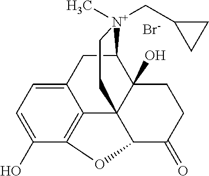 [SMILES: C[N+]1(CC2CC2)CC[C@]23c4c5ccc(O)c4O[C@H]2C(=O)CC[C@@]3(O)[C@H]1C5 ; [Br-]] | |
| 8-ketomethylnaltrexone bromide | RRT 0.49 |
| 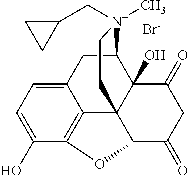 [SMILES: C[N+]1(CC2CC2)CC[C@]23c4c5ccc(O)c4O[C@H]2C(=O)CC(=O)[C@@]3(O)[C@H]1C5 ; [Br-]] | |
| Aldol dimer (dibromide) | RRT 1.77 |
| 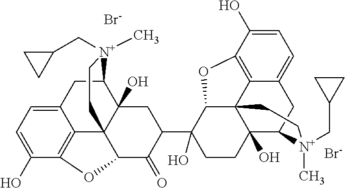 [SMILES: C[N+]1(CC2CC2)CC[C@]23c4c5ccc(O)c4O[C@H]2C(=O)C(C2(O)CC[C@@]4(O)[C@H]6Cc7ccc(O)c8c7[C@@]4(CC[N+]6(C)CC4CC4)[C@H]2O8)C[C@@]3(O)[C@H]1C5 ; [Br-] ; [Br-]] | |
| O-methyl Methylnaltrexone bromide | RRT 1.66 |
| (3-methyoxy naltrexone methobromide) | |
| 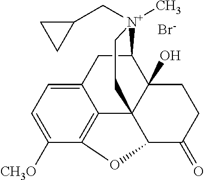 | |
| 2,2,bis-methylnaltrexone dibromide | RRT 1.55 |
| 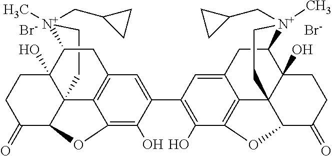 [SMILES: C[N+]1(CC2CC2)CC[C@]23c4c5cc(-c6cc7c8c(c6O)O[C@H]6C(=O)CC[C@@]9(O)[C@@H](C7)[N+](C)(CC7CC7)CC[C@]869)c(O)c4O[C@H]2C(=O)CC[C@@]3(O)[C@H]1C5 ; [Br-] ; [Br-]] | |

Naltrexone base, S-methylnaltrexone, and O-methyl Methylnaltrexone are each compounds found in initial production samples. Additional impurities/degradants formed and identified in methylnaltrexone formulations include 8-ketomethylnaltrexone bromide (RRT 0.49), the aldol dimer (RRT 1.77), O-methyl methylnaltrexone (RRT 1.66), and the 2,2 bis-methylnaltrexone (RRT 1.55), as well as additional degradants resulting at relative retention time of 0.67, 0.79 and 2.26.

Each of the three additional degradants were identified by NMR analysis following isolation from column eluates, and further characterized as described herein. The 0.67 degradant has been identified as 7-dihydroxy methylnaltrexone; the 0.79 degradant has been identified as a ring contracted form ((3R,4R,4aS,6aR,11bS)-6-carboxy-3-(cyclopropylmethyl)-4-a,6,8-trihydroxy-3-methyl-1,2,3,4,4a,5,6,6a-octahydro-4,11-methano[1]benzofuro[3′,2′:2,3]cyclopenta[1,2-c]pyridin-3-ium); and the 2.26 degradant has been identified as a Hoffman elimination product (see the following compound names, relative retention times, and associated structure).

| | |
|---|---|
| 7-dihydroxy methylnaltrexone bromide | RRT 0.67 |
| 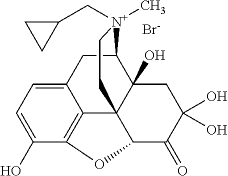 [SMILES: C[N+]1(CC2CC2)CC[C@]23c4c5ccc(O)c4O[C@H]2C(=O)C(O)(O)C[C@@]3(O)[C@H]1C5 ; [Br-]] | |
| ring contraction product | RRT 0.79 |
| (3R,4R,4aS,6aR,11bS)-6-carboxy- | |
| 3-(cyclopropylmethyl)-4a,6,8- | |
| trihydroxy-3-methyl-1,2,3,4,4a,5,6,6a- | |
| octahydro-4,11-methano[1] | |
| benzofuro[3′,2′:2,3] | |
| cyclopenta[1,2-c]pyridin-3-ium) | |
| 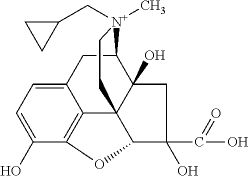 [SMILES: C[N+]1(CC2CC2)CC[C@]23c4c5ccc(O)c4O[C@H]2C(O)(C(=O)O)C[C@@]3(O)[C@H]1C5] | |
| Hofmann elimination product | RRT 2.26 |
| 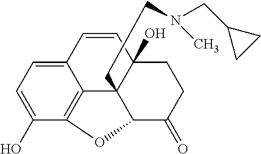 | |

Table 2 summarizes the formulation stability data from the high concentration methylnaltrexone formulation (24 mg/vial) at room temperature or 40° C./75% relative humidity from initial preparation until after 28 days of storage of sample. The data confirm that a lyophilized formulation consisting of methylnaltrexone and a single filler or single cryoprotectant remains stable, with total degradant formation remaining below 0.3% after 28 days of storage conditions. Furthermore, no degradant formation beyond those seen at initial preparation accumulated after 28 days of storage. Each of the peaks resulting in the NMR are represented in the table. For those products identified by the peaks: RRT 0.89 represents S-MNTX; RRT 1.17 represents Naltrexone Base; RRT 1.55 represent 2,2 Bis methylnaltrexone; RRT 1.66 represents O-Methyl-methylnaltrexone; RRT 1.77 represents aldol dimmer formation; RRT 2.26 represents the Hoffman elimination product. Tables 2A and 2B summarize stability data for a 24 mg/vial formulation until 6 or 12 months. Tables 2C and 2D summarize stability data for a 12 mg/vial formulation until 6 or 12 months.

Table 3 summarizes the formulation light stability data from middle concentration methylnaltrexone formulation (12 mg/vial) after storage of sample under dark or light conditions. The data confirms that a lyophilized formulation consisting of methylnaltrexone and cryoprotectant remains stable, with total degradant formation remaining below 0.12% after storage in light exposure.

TABLE 2
Stability of Lyophilized Methylnaltrexone Formulation

| | Initial | RRT | RRT | RRT | RRT | RRT | RRT | RRT | RRT | RRT | RRT | RRT | RRT | RRT | | Water |
|---|---|---|---|---|---|---|---|---|---|---|---|---|---|---|---|---|
| | (mg) | 0.38 | 0.49 | 0.67 | 0.79 | 0.89 | 1.17 | 1.55 | 1.66 | 1.77 | 1.89 | 1.96 | 2.01 | 2.26 | Total | Content |
| Initial | 24.8 | BRL | BRL | BRL | BRL | BRL | BRL | BRL | 0.11 | BRL | BRL | BRL | BRL | BRL | 0.18 | 1.63 |

Room Temperature

| | | | | | | | | | | | | | | | |
|---|---|---|---|---|---|---|---|---|---|---|---|---|---|---|---|
| Time and | | | | | | | | | | | | | | | |
| Days | | | | | | | | | | | | | | | |
| 10 | 24 | BRL | BRL | BRL | BRL | BRL | BRL | BRL | 0.1 | BRL | BRL | BRL | BRL | BRL | 0.24 |
| | (96.6) | | | | | | | | | | | | | | |
| 28 | 23.9 | BRL | BRL | BRL | BRL | BRL | BRL | BRL | 0.12 | BRL | BRL | BRL | BRL | BRL | 0.28 |
| | (96.2) | | | | | | | | | | | | | | |

40° C./75% Relative Humidity

| | | | | | | | | | | | | | | | | |
|---|---|---|---|---|---|---|---|---|---|---|---|---|---|---|---|---|
| 7 | | | | | | | | | | | | | | | | 1.91 |
| 10 | 23.9 | BRL | BRL | BRL | BRL | BRL | BRL | BRL | 0.12 | BRL | BRL | BRL | BRL | 0.05 | 0.24 | |
| | (96.2) | | | | | | | | | | | | | | | |
| 18 | 24.4 | BRL | BRL | BRL | BRL | BRL | BRL | BRL | 0.12 | BRL | BRL | BRL | BRL | 0.08 | 0.27 | |
| | (98.2) | | | | | | | | | | | | | | | |
| 28 | 23.9 | BRL | BRL | BRL | BRL | BRL | BRL | BRL | 0.13 | BRL | BRL | BRL | BRL | 0.07 | 0.28 | |
| | (96.2) | | | | | | | | | | | | | | | |

TABLE 2A
Stability of Methylnaltrexone 24 mg/vial Lyophilized Powder for Infusion

| | Description | Description | | | | | |
|---|---|---|---|---|---|---|---|
| Storage | Lyophilized | Reconstituted | | | | | |
| Time | Powder | Solution | pH | Water Content | Particulate Matter | | |
| Standards | White to pale | Clear, colorless to | 3.5-7.5 | NMT 5.0% | NMT 6000 | NMT 600 | Meet |
| | yellow cake plug | pale yellow | | | particles | particles | USP <788> |
| | or powdered cake | solution, essentially | | | ≧ 10 microns | ≧ 25 microns | criteria |
| | | free of visible | | | | | |
| | | particulates | | | | | |
| Method | HPLC | HPLC | USP | USP <921>lc | USP <788> | | |
| | | | <791> | | | | |
| Initial | Conforms | Conforms | 5.1, 5.1 | 0.71 | 20 | 1 | Conforms |

Storage Condition 25° C./60% RH

| | | | | | | | |
|---|---|---|---|---|---|---|---|
| 1 Month | Conforms | Conforms | 5.2, 5.2 | 1.50 | 17 | 0 | Conforms |
| 3 Months | Conforms | Conforms | 5.3, 5.4 | 2.52 | 32 | 1 | Conforms |
| 6 Months | Conforms | Conforms | 5.3, 5.3 | 2.75 | 13 | 0 | Conforms |
| 9 Months | Conforms | Conforms | 5.2, 5.2 | 2.68 | 12 | 1 | Conforms |
| 12 Months | Conforms | Conforms | 6.2, 6.1 | 3.08 | 12 | 1 | Conforms |

Storage Condition 30° C./75% RH

| | | | | | | | |
|---|---|---|---|---|---|---|---|
| 1 Month | Conforms | Conforms | 5.2, 5.2 | 3.24 | NS | NS | NA |
| 6 Months | Conforms | Conforms | 5.3, 5.3 | 2.98 | NS | NS | NA |

Storage Condition 40° C./75% RH

| | | | | | | | |
|---|---|---|---|---|---|---|---|
| 1 Month | Conforms | Conforms | 5.2, 5.3 | 2.17 | NS | NS | NA |
| 3 Months | Conforms | Conforms | 5.4, 5.4 | 3.29 | NS | NS | NA |
| 6 Months | Conforms | Conforms | 5.4, 5.4 | 2.74 | NS | NS | NA |

TABLE 2B
Stability of Methylnaltrexone 24 mg/vial Lyophilized Powder for Infusion

| | | Degradation/Impurities | | | | | | | | |
|---|---|---|---|---|---|---|---|---|---|---|
| | | 7- | | | | | | | Largest | Total |
| Storage | | Dihydroxy | Ring | Naltrexone | 2, 2′-bis | O-Methyl^{a} | Aldol- | Hofmann | Single | Degradants/ |
| Time | Strength | MNTX | Contraction | Base | MNTX | MNTX | Dimer | Elimination | Unspecified | Impurities |
| Standards | 95.0- | NMT 0.5% | NMT 0.5% | FIO | NMT | FIO | NMT | NMT 0.5% | NMT 0.2% | NMT 2.0% |
| | 115.0% | w/w | w/w | | 0.5% | | 0.5% | w/w | w/w | w/w |
| | LC | | | | w/w | | w/w | | | |
| Initial | 104.4 | BRL | BRL | BRL | BRL | BRL | BRL | BRL | BRL | BRL |

Storage Condition 25° C./60% RH

| | | | | | | | | | | |
|---|---|---|---|---|---|---|---|---|---|---|
| 1 Month | 104.1 | BRL | BRL | BRL | BRL | BRL | BRL | BRL | BRL | BRL |
| 3 Months | 104.5 | BRL | BRL | BRL | BRL | BRL | BRL | BRL | BRL | BRL |
| 6 Months | 104.8 | BRL | BRL | BRL | BRL | BRL | BRL | BRL | BRL | BRL |
| 9 Months | 105.3 | BRL | BRL | BRL | BRL | BRL | BRL | BRL | BRL | BRL |
| 12 Months | 105.5 | BRL | BRL | BRL | BRL | BRL | BRL | BRL | BRL | BRL |

Storage Condition 30° C./75% RH

| | | | | | | | | | | |
|---|---|---|---|---|---|---|---|---|---|---|
| 1 Month | 105.0 | BRL | BRL | BRL | BRL | BRL | BRL | BRL | BRL | BRL |
| 6 Months | 106.2 | BRL | BRL | BRL | BRL | BRL | BRL | BRL | BRL | BRL |

Storage Condition 40° C./75% RH

| | | | | | | | | | | |
|---|---|---|---|---|---|---|---|---|---|---|
| 1 Month | 103.5 | BRL | BRL | BRL | BRL | BRL | BRL | BRL | 0.08 | 0.08 |
| 3 Months | 103.6 | BRL | BRL | BRL | BRL | BRL | BRL | BRL | BRL | BRL |
| 6 Months | 107.0 | BRL | BRL | BRL | BRL | BRL | BRL | BRL | BRL | BRL |

BRL = Below reporting limit (0.05%)
NA = Not applicable
NS = Not scheduled
LC = Label claim
NMT = Not more than
RRT = Relative retention time
FIO = For information only.

TABLE 2C
Stability of Lyophilized Methylnaltrexone Formulation
Stability of Methylnaltrexone 12 mg/vial Lyophilized Powder for Infusion

| | Description | Description | | | | | |
|---|---|---|---|---|---|---|---|
| Storage | Lyophilized | Reconstituted | | | | | |
| Time | Powder | Solution | pH | Water Content | Particulate Matter | | |
| Standards | White to pale | Clear, colorless to | 3.5-7.5 | NMT 5.0% | NMT 6000 particles | NMT 600 particles | Meet USP <788> |
| | yellow cake plug | pale yellow | | | ≧10 microns | ≧25 microns | criteria |
| | or powdered | solution, essentially | | | | | |
| | cake | free of visible | | | | | |
| | | particulates | | | | | |
| Method | HPLC | HPLC | USP | USP <921> Ic | USP <788> | | |
| | | | <791> | | | | |
| Initial | Conforms | Conforms | 5.0, 5.1 | 0.80 | 12 | 2 | Conforms |

Storage Condition 25° C./60% RH

| | | | | | | | |
|---|---|---|---|---|---|---|---|
| 1 Month | No Change | No Change | 5.2, 5.1 | 1.47 | 96 | 4 | Conforms |
| 3 Months | No Change | No Change | 5.4, 5.3 | 2.10 | 249 | 10 | Conforms |
| 6 Months | No Change | No Change | 5.4, 5.4 | 2.24 | 16 | 1 | Conforms |
| 9 Months | No Change | No Change | 5.2, 5.2 | 2.77 | 24 | 3 | Conforms |
| 12 Months | No Change | No Change | 5.3, 5.3 | 2.83 | 13 | 3 | Conforms |

Storage Condition 30° C./75% RH

| | | | | | | | |
|---|---|---|---|---|---|---|---|
| 1 Month | No Change | No Change | 5.2, 5.3 | 3.59 | NS | NS | NA |
| 6 Months | No Change | No Change | 5.4, 5.4 | 3.37 | NS | NS | NA |

Storage Condition 40° C./75% RH

| | | | | | | | |
|---|---|---|---|---|---|---|---|
| 1 Month | No Change | No Change | 5.0, 5.0 | 2.26 | NS | NS | NA |
| 3 Months | No Change | No Change | 5.4, 5.3 | 3.31 | NS | NS | NA |
| 6 Months | No Change | No Change | 5.4, 5.4 | 3.18 | NS | NS | NA |

TABLE 2D
Stability of Methylnaltrexone 12 mg/vial Lyophilized Powder for Infusion

| | | Degradation/Impurities | | | | | | | | |
|---|---|---|---|---|---|---|---|---|---|---|
| | | 7- | | | | | | | Largest | Total |
| Storage | | Dihydroxy | Ring | Naltrexone | 2,2′-bis | O-Methyl^{a} | Aldol- | Hofmann | Single | Degradants/ |
| Time | Strength | MNTX | Contraction | Base | MNTX | MNTX | Dimer | Elimination | Unspecified | Impurities |
| Standards | 95.0-115.0% | NMT | NMT | FIO | NMT | FIO | NMT | NMT | NMT | NMT |
| | LC | 0.5% | 0.5% | | 0.5% | | 0.5% | 0.5% | 0.2% | 2.0% |
| | | w/w | w/w | | w/w | | w/w | w/w | w/w | w/w |
| Initial | 103.5 | BRL | BRL | BRL | BRL | BRL | BRL | BRL | BRL | BRL |

Storage Condition 25° C./60% RH

| | | | | | | | | | | |
|---|---|---|---|---|---|---|---|---|---|---|
| 1 Month | 102.6 | BRL | BRL | BRL | BRL | BRL | BRL | BRL | 0.06 | 0.06 |
| | | | | | | | | | (RRT 0.89) | |
| 3 Months | 102.6 | BRL | BRL | BRL | BRL | BRL | BRL | BRL | BRL | BRL |
| 6 Months | 103.1 | BRL | BRL | BRL | BRL | BRL | BRL | BRL | BRL | BRL |
| 9 Months | 104.9 | BRL | BRL | BRL | BRL | BRL | BRL | BRL | BRL | BRL |
| 12 Months | 104.1 | BRL | BRL | BRL | BRL | BRL | BRL | BRL | BRL | BRL |

Storage Condition 30° C./75% RH

| | | | | | | | | | | |
|---|---|---|---|---|---|---|---|---|---|---|
| 1 Month | 104.3 | BRL | BRL | BRL | BRL | BRL | BRL | BRL | BRL | BRL |
| 6 Months | 105.3 | BRL | BRL | BRL | BRL | BRL | BRL | BRL | BRL | BRL |

Storage Condition 40° C./75% RH

| | | | | | | | | | | |
|---|---|---|---|---|---|---|---|---|---|---|
| 1 Month | 101.8 | BRL | BRL | BRL | BRL | BRL | BRL | BRL | 0.07 | 0.07 |
| | | | | | | | | | (RRT 0.89) | |
| 3 Months | 101.8 | BRL | BRL | BRL | BRL | BRL | BRL | BRL | BRL | BRL |
| 6 Months | 103.8 | BRL | BRL | BRL | BRL | BRL | BRL | BRL | BRL | BRL |

BRL = Below reporting limit (0.05%)
NA = Not applicable
NS = Not scheduled
NMT = Not more than
RRT = Relative retention time
FIO = For information only.

TABLE 3
Light Stability Of Methylnaltrexone Lyophilized
Powder for injection (12 MG/VIAL)

| Storage | Initial | RRT | RRT | RRT | RRT | RRT | RRT | RRT | RRT | RRT | RRT |
|---|---|---|---|---|---|---|---|---|---|---|---|
| time | (mg) | 0.60 | 0.63 | 0.67 | 0.70 | 0.79 | 0.84 | 0.89 | 1.17 | 1.3 | 1.47 |
| Time | 12.35 | BRL | BRL | BRL | BRL | BRL | BRL | BRL | BRL | BRL | BRL |
| Zero | | | | | | | | | | | |
| Light | 12.24 | BRL | BRL | BRL | BRL | BRL | BRL | BRL | BRL | BRL | BRL |
| ICH2 | | | | | | | | | | | |
| Storage | RRT | RRT | RRT | RRT | RRT | RRT | RRT | RRT | RRT | RRT | |
| time | 1.52 | 1.55) | 1.58 | 1.66 | 1.77 | 1.81 | 1.89 | 1.91 | 2.11 | 2.26 | Total |
| Time | BRL | BRL | BRL | 0.12 | BRL | BRL | BRL | BRL | BRL | BRL | 0.12 |
| Zero | | | | | | | | | | | |
| Light | BRL | BRL | BRL | 0.12 | BRL | BRL | BRL | BRL | BRL | BRL | 0.12 |
| ICH2 | | | | | | | | | | | |

BRL = Below Reporting Limit of 0.05%

### Example 3

In certain embodiments, the present invention provides a methylnaltrexone formulation for intravenous administration. Provided intravenous formulations can be prepared in 12 mg/vial or 24 mg/vial concentrations. Both 12 mg/vial and 24 mg/vial strengths use a 5 mg/mL concentration of methylnaltrexone. In certain embodiments, provided intravenous formulations utilize a 10 mL spikable vial designed to be used with Baxter mini-bags or any other spikable infusion system. In some embodiments, provided formulations were subjected to terminal sterilization by heating at 121° C. for 15 minutes.

In certain embodiments, formulations are prepared in 12 mg/vial or 24 mg/vial concentrations. Such formulations can be administered at doses of 24 mg, or also, for example, 0.3 mg/kg, every 6 hours as a 20-minute infusion. In certain embodiments, such administration is continued for 3 days (total of 12 doses). Each methylnaltrexone formulation is diluted to 50 mL and administered using a calibrated pump.

In certain embodiments, fill volume is at least 2.6 mL for a 2.4 mL extractable volume, and at least 5.1 mL for a 4.8 mL extractable volume. Table 5 below describes vial contents dilution when using a traditional syringe or a spikable vial.

TABLE 5
Overage and Reconstitution of Sample

| | spikable technique with | | traditional syringe | |
|---|---|---|---|---|
| | Baxter Mini-bag | | withdrawal | |
| Concentration | 5 mg/mL | 5 mg/mL | 5 mg/mL | 5 mg/mL |
| mg/vial | 12 mg | 24 mg | 12 mg | 24 mg |
| Overage | 5% | 5% | 5% | 5% |
| Fill volume | 2.52 | 5.04 | 2.52 | 5.04 |
| Reconstitution | 8.0 mL | 5.0 mL of saline | 8.0 mL of | 5.0 mL of saline solution |
| volume | of saline | solution | saline | |
| | solution | | solution | |
| Withdrawal | Spike | Spike full | Withdraw | Withdraw 10.0 mL via |
| amount | full | contents of vial | 10.0 mL | syringe |
| | contents | | via | |
| | of vial | | syringe | |

### Example 4

In certain embodiments, a provided intravenous formulation is administered to a patient 90 minutes post surgery, where the surgery is hernia repair. In some embodiments, the hernia repair patient is administered opioids via PCA pump. Such formulations can be administered at doses of 12 mg or 24 mg, or also, for example, 0.3 mg/kg, every 6 hours as a 20-minute infusion. In certain embodiments, such administration is continued for 10 days, the patient is discharged, or 24 hours post-bowel movement.

### EQUIVALENTS

One skilled in the art will readily ascertain the essential characteristics of the invention, and understand that the foregoing description and examples are illustrative of practicing the provided invention. Those skilled in the art will be able to ascertain using no more than routine experimentation, many variations of the detail presented herein may be made to the specific embodiments of the invention described herein without departing from the spirit and scope of the present invention.

Patents, patent applications, publications, and the like are cited throughout the application. The disclosures of each of these documents are incorporated herein by reference in their entirety.

We claim:

1. An amorphous dry powder formulation consisting essentially of methylnaltrexone, or a pharmaceutically acceptable salt thereof, and a filler.

2. The formulation of claim 1, wherein the methylnaltrexone is methylnaltrexone bromide.

3. The formulation of claim 1, wherein the filler is selected from the group consisting of a lactose, mannitol, and dextran.

4. The formulation of claim 3, wherein the filler is a lactose and the methylnaltrexone is methylnaltrexone bromide.

5. The formulation of claim 4, wherein the lactose is lactose monohydrate.

6. The formulation of claim 1, consisting essentially of:
about 5 to about 500 mg of methylnaltrexone bromide; and
lactose monohydrate.

7. The formulation of claim 1, wherein the methylnaltrexone and filler are present in approximately equal amounts by weight.

8. The formulation of claim 1, wherein the methylnaltrexone and filler are present in a ratio within the range of about 1:1 to about 1:5 by weight.

9. A solution consisting essentially of water and the formulation of claim 4.

10. The solution of claim 9, wherein the lactose is lactose monohydrate.

11. The solution of claim 10, wherein the methylnaltrexone bromide and lactose monohydrate are present in approximately equal amounts by weight.

12. The solution of claim 11, wherein methylnaltrexone bromide is present in a concentration of about 0.5 mg/mL to about 25 mg/mL.

13. A method of producing a stable, sterile pharmaceutical product comprising steps of:
obtaining the solution of claim 9; and
lyophilizing the composition.

14. A pharmaceutical dosage preparation comprising an amorphous solid pharmaceutical formulation consisting essentially of methylnaltrexone, or a pharmaceutically acceptable salt thereof, and a filler, in a sealed container.

15. The pharmaceutical dosage preparation of claim 14, wherein the filler is selected from the group consisting of a lactose, mannitol, and dextran.

16. The pharmaceutical dosage preparation of claim 15, wherein the filler is a lactose.

17. The pharmaceutical dosage preparation of claim 15, wherein the lactose is lactose monohydrate.

18. A method for reducing the side effects of opioid therapy in a subject receiving opioid treatment or use comprising administering to a subject in need thereof a regimen comprising reconstituting a formulation according to claim 1 in a pharmaceutically acceptable aqueous solvent, and administering the solution to the subject.

19. The method according to claim 18, wherein the reconstituting step is followed by diluting the reconstituted formulation in an isotonic carrier, and administering the diluted solution to the subject.

20. A kit comprising a first container containing a formulation according to claim 1, and a second container containing an aqueous carrier.
